(19) United States
(12) Patent Application Publication
(10) Pub. No.: US 2026/0242746 A1
(43) Pub. Date: Aug. 20, 2026

(54) METHODS OF CELL PRESERVATION

(71) Applicant: Be Biopharma, Inc., Cambridge, MA (US)

(72) Inventors: Mark Louis Lalli, Medford, MA (US); Jordan Marie Smelker, Medford, MA (US)

(21) Appl. No.: 19/156,290

(22) PCT Filed: Feb. 14, 2024
(86) PCT No.: PCT/US24/15731

§ 371 Date: Aug. 13, 2025

Related U.S. Application Data
(60) Provisional application No. 63/445,690, filed on Feb. 14, 2023.

(51) Int. Cl. C12N 5/00; A61K 35/17

(52) U.S. Cl. CPC C12N 5/525; A61K 35/17

(57) ABSTRACT

The present disclosure provides methods and compositions for culturing, cryopreserving, and administering engineered B lineage cell populations. The methods and compositions are provided herein.

### CROSS-RELATED TO RELATED APPLICATIONS

This application claims priority to U.S. Provisional Application No. 63/445,690, filed on Feb. 14, 2023, the entirety of which is incorporated herein by reference.

### BACKGROUND

Cell therapy is an emerging from of treatment that comprises administration of viable human cells to a subject to act as “living drugs.” In particular, certain cell therapies make use of engineered immune cells, to combat disease. Maintenance and cryopreservation of these engineered adaptive immune cells are critical for eventual administration both in clinical and pre-clinical settings.

The present disclosure provides, inter alia, methods of cryopreservation, culturing, and administration of engineered immune cells.

### SUMMARY

Among other things, the present disclosure identifies challenges in providing engineered cell preparations for treatment of disease, as storage and transport of cell preparations can lead to cell death, stress response, and/or loss of transgene expression. In some embodiments, the present disclosure provides methods for cryopreservation of B lineage cell preparations. In some embodiments, the present disclosure provides methods for cryopreservation of engineered B lineage cell preparations. In particular, the present disclosure provides methods for cryopreservation of engineered B lineage cell preparations to maintain or prevent significant reduction of cell viability and/or transgene expression. In some embodiments, the present disclosure provides pharmaceutical compositions comprising engineered B lineage cell preparations that maintain or do not have significant reduction in engraftment and/or transgene expression after administration to a subject.

The present disclosure encompasses the recognition of a problem with cryopreservation and eventual thawing of B lineage cell populations. For example, cooling of cell populations and associated aqueous solution present formation of excessive intracellular ice formation, induction of cellular dehydration, potential deleterious changes in cell morphology (e.g., adhesion, cell surface markers) and metabolism (e.g., potency), and reduced viability (See Meneghel, et al. 2020, incorporated by reference herein in its entirety).

Among other things, in one aspect, the present disclosure provides methods of preparing a B lineage cell population for cryopreservation, the method comprising a step of: contacting the B lineage cell population with cryopreservation media comprising about 4.5% to about 5% DMSO and about 2.5% to about 10% HSA thereby producing a cryopreservation cell preparation.

In some embodiments, a cryopreservation cell preparation undergoes controlled cooling in a controlled cooling chamber, comprising one or more steps of: (i) cooling a chamber until the cryopreservation cell preparation reaches 4° C.; (ii) cooling a chamber at a rate of 1° C./minute until a cryopreservation cell preparation reaches −4° C.; (iii) cooling a chamber at a rate of 25° C. /minute until a chamber reaches −40° C.; (iv) heating a chamber at a rate of 10° C. /minute until a chamber reaches −12° C.; (v) cooling a chamber at a rate of 1° C./minute until a chamber reaches −40° C.; (vi) cooling a chamber at a rate of 10° C./minute until a chamber reaches −90° C.

In some embodiments, a B lineage cell population is or comprises a population of genetically modified plasma cells. In some embodiments, a B lineage cell population is or comprises a population of plasmablasts. In some embodiments a B lineage cell population is or comprises a population of plasma cell precursors.

In some embodiments, a cryopreservation cell preparation maintains at least 50% viability after being frozen for at least 24 hours. In some embodiments, a cryopreservation cell preparation maintains at least 50% viability after being frozen for one week. In some embodiments, a cryopreservation cell preparation maintains at least 50% viability after being frozen for one month. In some embodiments, a cryopreservation cell preparation maintains at least 50% viability after being frozen for six months.

In another aspect, the present disclosure provides compositions comprising a population of frozen, genetically modified B lineage cells, and cryopreservation media comprising: about 4.5% to about 5% DMSO and about 2.5% to about 10% HSA. In some embodiments, a cryopreservation media comprises 5% DMSO. In some embodiments, a cryopreservation media comprises 2.5% HSA.

In some embodiments, a composition of B lineage cells are genetically modified with a transgene in an endogenous gene locus. In some embodiments, the transgene is Factor IX. In some embodiments, an endogenous gene locus is CCR5 or JCHAIN.

In some embodiments, a genetically modified B lineage cells have been frozen for at least 24 hours and maintain at least 50% viability after thawing. In some embodiments, a genetically modified B lineage cells have been frozen for at least 24 hours and at least 25% of cells secrete IgG after thawing. In some embodiments, a genetically modified B lineage cells have been frozen for at least 24 hours and at least 25% of cells express IgM after thawing.

In some embodiments, a genetically modified B lineage cell population has been frozen for at least one week. In some embodiments, a genetically modified B lineage cell population has been frozen for at least one month. In some embodiments, a genetically modified B lineage cell population has been frozen for at least six months.

In another aspect, the present disclosure provides methods of treating for a disease that comprises administering a population of genetically modified B lineage cells that have been cryopreserved. In some embodiments, a population of genetically modified B lineage cells have been cryopreserved in media comprising about 4.5% to about 5% DMSO and about 2.5% to about 10% HSA and thawed prior to administration to a patient. In some embodiments, a population of genetically modified B lineage cells have been cryopreserved in media comprising 5% DMSO. In some embodiments, a population of genetically modified B lineage cells have been cryopreserved in media comprising 2.5% HSA.

### BRIEF DESCRIPTION OF THE DRAWING

FIG. 1 shows measurements of bioluminescence in mice after engraftment of engineered B lineage cell preparations expressing luciferase (“Luc”) at the CCR5 locus. Both fresh B lineage cell preparations and sub-optimal cryopreserved B lineage cell preparations were administered. Conditions tested from left to right are: PBS only, LKP13 Culture Condition 1 Cryo, LKP13 Culture Condition 1 Fresh, LKP42 Culture Condition 1 Cryo, and LKP42 Culture Condition 1 Fresh.

FIG. 2 shows bioluminescence measurements of whole mice after administration and engraftment of B lineage cell preparations and PBS control.

FIG. 3 shows levels of human IgG in the plasma of mice after engraftment of engineered B lineage cell preparations. Conditions tested from left to right are: PBS only, LKP13 Culture Condition 1 Cryo, LKP13 Culture Condition 1 Fresh, LKP42 Culture Condition 1 Cryo, and LKP42 Culture Condition 1 Fresh.

FIG. 4 shows a comparison measurement of cell viability for cryopreserved B lineage cell preparations, fresh engineered B lineage cell preparations (“Fresh Cells”), and failed cryopreserved engineered B lineage cell preparations that were engrafted in a mouse model (“LKP13 Culture Condition 1 Cryo” and “LKP42 Culture Condition 1 Cryo”). Cell preparations were cultured for 13 days, then cryopreserved and thawed at indicated times.

FIG. 5 shows comparison measurements of cell viability for previously frozen engineered B lineage cell preparations (“LKP13 Cryo” and “LKP42 Cryo”), fresh engineered B lineage cell preparations, and cryopreserved engineered B lineage cell preparations prepared by the methods described herein.

FIG. 6 shows grouped and individual bioluminescence measurements over time for mice after administration of either indicated fresh engineered B lineage cell preparations or cryopreserved engineered B lineage cell preparations, with bioluminescence measured at indicated times. Conditions tested from left to right are: untreated, LKP76 Culture Condition 1 Fresh, LKP76 Culture Condition 1 Cryo, LKP76 Culture Condition 2 Fresh, LKP76 Culture Condition 2 Cryo, LKP60 Culture Condition 1 Fresh, and LKP60 Culture Condition 1 Cryo.

FIG. 7 shows a comparison measurement of viable cell density (VCD) pre-freeze (left bar of pairings) and post-thaw (right bar of pairings) for numerous engineered B lineage cell preparations.

FIG. 8 shows a comparison measurement of cell viability for engineered B lineage cell preparations pre-freeze (left bar of pairings) and post-thaw (right bar of pairings).

FIG. 9 demonstrates viability and percentage of an engineered B lineage cell preparation that is CD27 positive, CD38 positive, CD27 and CD38 positive, and Factor IX (F9) transgene insertion before (Time 0) and during undergoing the cryopreservation methods described herein.

FIG. 10 shows measurements of IgG and IgM over time for mice after administration of indicated fresh engineered B lineage cell preparations or cryopreserved engineered B lineage cell preparations using the methods disclosed herein.

FIG. 11 shows measurements of human Factor IX (huFIX) over time for mice after administration of B lineage cell population that were engineered and preserved with the methods as disclosed herein.

FIG. 12A-B shows measurements of human Factor IX (huFIX) and human immunoglobulin G (IgG) over time for mice after administration of four donors of B lineage cell populations (LKP23008, LKP22090, LKP22094, and LKP22091) that were engineered and preserved with the methods as disclosed herein.

FIG. 13A-B shows measurements of huFIX, IgG, and human immunoglobulin M (huIgM) over time for mice after administration and re-dosing at day 21 with engineered B lineage cell population that were preserved with the methods as disclosed herein.

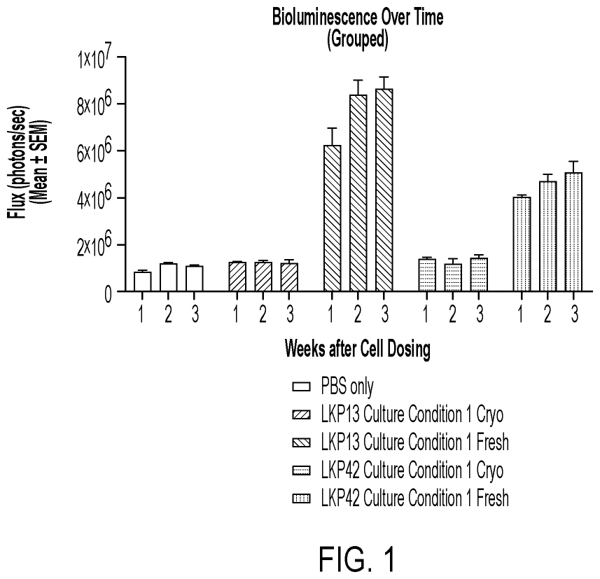

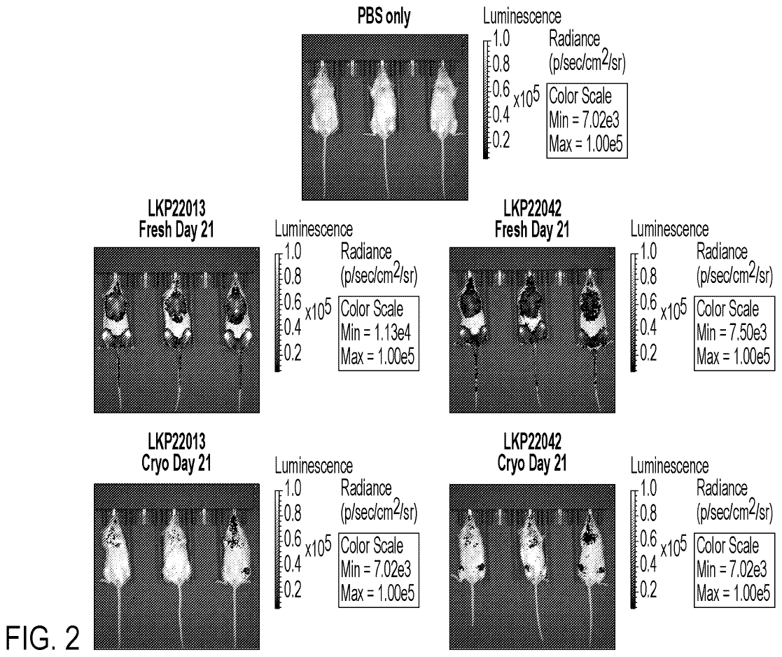

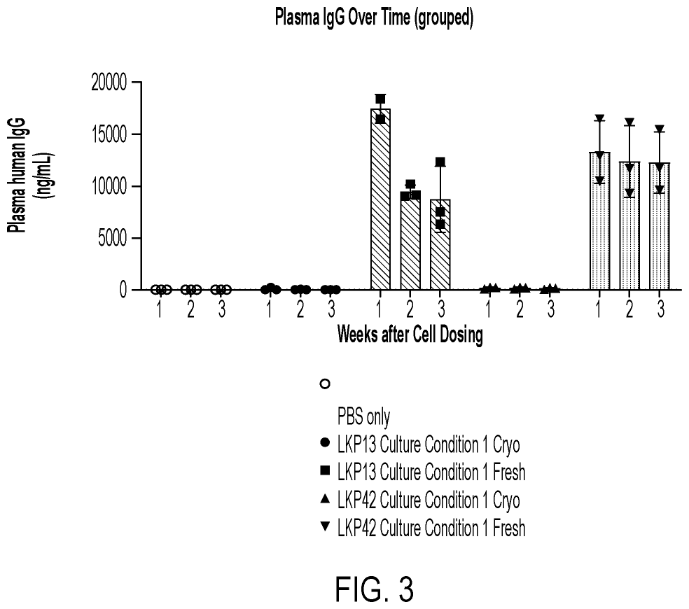

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Activating agent: As used herein, the term “activating agent” refers to an agent whose presence or level correlates with elevated level or activity of a target, as compared with that observed absent the agent (or with the agent at a different level). In some embodiments, an activating agent is one whose presence or level correlates with a target level or activity that is comparable to or greater than a particular reference level or activity (e.g., that observed under appropriate reference conditions, such as presence of a known activating agent, e.g., a positive control).

Administration: As used herein, the term “administration” typically refers to the administration (e.g., of a composition or treatment) to a subject or system (e.g., that is or comprises one or more cells, tissues, organisms, etc), for example to achieve delivery of an agent that is, is included in, or is otherwise delivered or generated by, such composition or treatment. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Alloantigen: The term “alloantigen”, as used herein, refers to an antigen associated with allorecognition and/or graft rejection (e.g., an antigen against which a rejection immune response is directed). In general, alloantigens are agents that are present in or on tissue from one individual (e.g., a donor individual) of a particular species, but not in or on tissue from another individual (e.g., a recipient individual, for example who is genetically different from the donor individual) of the species, so that transfer of tissue from the donor individual to the recipient individual risks and/or results in a rejection immune response. In general, an antigen may be or include any chemical entity such as, for example, a small molecule, a nucleic acid, a polypeptide, a carbohydrate, a lipid, etc. In some embodiments, an alloantigen is or comprises a polypeptide. A variety of polypeptides are known in the art whose amino acid sequences can vary between and among individuals of the same species such that they might act as alloantigens.

Allorecognition: The term “allorecognition”, as used herein, typically refers to an immune response mounted by the immune system of an individual (i.e., a recipient) who receives a tissue graft from another individual (i.e., a donor, who for example is genetically distinct from the recipient individual) of the same species, which immune response involves recognition of an alloantigen on the grafted tissue. Typically, allorecognition involves T cell recognition of the alloantigen. In many embodiments, T cells recognize an alloantigen peptide, for example, encoded by a polymorphic gene whose sequence differs between the donor and recipient individuals.

Amelioration: As used herein, refers to the prevention, reduction or palliation of a state, or improvement of the state of a subject. Amelioration includes, but does not require complete recovery or complete prevention of a disease, disorder or condition (e.g., radiation injury).

Antigen: As used herein, the term “antigen” or “Ag” refers to a molecule that is capable of provoking an immune response. This immune response may involve either antibody production, activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Antibody agent: As used herein, the term “antibody agent” (interchangeably referred to herein as “antibody”) refers to a polypeptide that may be expressed, released, secreted, or delivered to a target by a modified cell described herein. The polypeptide includes canonical immunoglobulin sequence elements sufficient to confer specific binding to a particular target antigen. In some embodiments, an antibody agent comprises of an antibody. As is known in the art, antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody agent tetramers comprises two heavy chain-light chain dimers in which the heavy and light chains are linked to one another by a single disulfide bond; two other disulfide bonds connect the heavy chain hinge regions to one another, so that the dimers are connected to one another and a tetramer is formed. Antibody agents are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present disclosure (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody agent”, whether such polypeptide is naturally produced (e.g., generated by an organism reacting to an antigen), or produced by recombinant engineering, chemical synthesis, or other artificial system or methodology. In some embodiments, an antibody agent is polyclonal. In some embodiments, an antibody agent is monoclonal. In some embodiments, an antibody agent has constant region sequences that are characteristic of mouse, rabbit, primate, or human antibodies. In some embodiments, antibody agent sequence elements are humanized, primatized, chimeric, etc, as is known in the art. Moreover, the term “antibody agent”, as used herein, can refer in appropriate embodiments (unless otherwise stated or clear from context) to any of the art-known or developed constructs or formats for utilizing antibody structural and functional features in alternative presentation. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc.].

Biologically active: As used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Biomarker: The term “biomarker” is used herein, consistent with its use in the art, to refer to a to an entity, event, or characteristic whose presence, level, degree, type, and/or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is detected outside of cells (e.g., is secreted or is otherwise generated or present outside of cells, e.g., in a body fluid such as blood, urine, tears, saliva, cerebrospinal fluid, etc. In some embodiments, a biomarker may be or comprise a genetic or epigenetic signature. In some embodiments, a biomarker may be or comprise a gene expression signature.

Bispecific antibody: As used herein, refers to a bispecific binding agent in which at least one, and typically both, of the binding moieties is or comprises an antibody component. A variety of different bi-specific antibody structures are known in the art. In some embodiments, each binding moiety in a bispecific antibody that is or comprises an antibody component includes V_{H} and/or V_{L} regions; in some such embodiments, such V_{H} and/or V_{L} regions are those found in a particular monoclonal antibody. In some embodiments, where the bispecific antibody contains two antibody component-binding moieties, each includes V_{H} and/or V_{L} regions from different monoclonal antibodies. In some embodiments, a bispecific antibody contains two antibody component binding moieties, wherein one of the two antibody component binding moieties includes an immunoglobulin molecule having V_{H} and/or V_{L} regions that contain CDRs from a first monoclonal antibody, and one of the two antibody component binding moieties includes an antibody fragment (e.g., Fab, F(ab′), F(ab′)₂, Fd, Fv, dAB, scFv, etc.) having V_{H} and/or V_{L} regions that contain CDRs from a second monoclonal antibody.

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Controlled cooling: As used here, the term “controlled cooling” (also interchangeably referred to herein as “controlled cooling rate”) may refer to a process of both freezing and heating a cell preparation. In some embodiments, controlled cooling may preserve structural and/or functional integrity of said cell preparation. Controlled cooling may also encompass specific steps and parameters wherein a cell preparation may be exposed to different temperatures, at different cooling rates and/or times to maximize viability and function of a cell preparation. In some embodiments, controlled cooling may occur in a controlled cooling chamber comprising one or more steps.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in an engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with a second coding sequence. Comparably, a polypeptide may be considered to be “engineered” if encoded by or expressed from an engineered polynucleotide, and/or if produced other than natural expression in a cell. Analogously, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, such manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Excipient: As used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to the generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Functional: As used herein, a “functional” biological molecule is a biological molecule in a form in which it exhibits a property and/or activity by which it is characterized.

Gene product or expression product: As used herein, the term “gene product” or “expression product” generally refers to an RNA transcribed from a gene (pre-and/or post-processing) or a polypeptide (pre-and/or post-modification) encoded by an RNA transcribed from a gene.

Isolated: As used herein, refers to a substance and/or entity that has been (1) separated from at least some of the components with which it was associated when initially produced (whether in nature and/or in an experimental setting) and/or otherwise previously associated, and/or (2) designed, produced, prepared, and/or manufactured by the hand of man. In some embodiments, a substance may be considered to be “isolated” if it is (or has been caused to be) free of or separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of other components (e.g., components with which it was previously associated). In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of a substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of presence or absence of a marker may vary depending upon a particular marker. In some embodiments, detection of a marker is highly specific in that it reflects a high probability that such tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. Those skilled in the art will appreciate that, in many embodiments, a useful marker need not distinguish with 100% accuracy.

Payload: In general, the term “payload”, as used herein, refers to an agent that may be delivered or transported by association with another entity. In some embodiments, such association may be or include a covalent linkage; in some embodiments such association may be or include non-covalent interaction(s). In some embodiments, association may be direct; in some embodiments, association may be indirect. The term “payload” is not limited to a particular chemical identity or type; for example, in some embodiments, a payload may be or comprise, for example, an entity of any chemical class including, for example, a lipid, a metal, a nucleic acid (e.g., a transgene), a polypeptide, a saccharide (e.g., a polysaccharide), small molecule, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, etc., or a combination thereof. In some embodiments, a payload may be or comprise a cell or organism, or a fraction, extract, or component thereof. In some embodiments, a payload may be or comprise a natural product in that it is found in and/or is obtained from nature; alternatively or additionally, in some embodiments, the term may be used to refer to one or more entities that is man-made in that it is designed, engineered, and/or produced through action of the hand of man and/or is not found in nature. In some embodiments, an payload may be or comprise an agent in isolated or pure form; in some embodiments, such agent may be in crude form.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a response to treatment may refer to a beneficial alteration in a subject's condition that occurs as a result of or correlates with treatment. In some embodiment, such alteration may be or comprise stabilization of a condition (e.g., prevention of deterioration that would have taken place in the absence of a treatment), amelioration of symptoms of a condition, and/or improvement in prospects for cure of a condition, etc. In some embodiments, the term “response” may refer to a response of a particular system or components thereof (e.g., of a particular cell, tissue, organism, or subject). Those skilled in the art will be aware of technologies available to assess a response of interest.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample may be a “crude” sample in that it has been subjected to relatively little processing and/or is complex in that it includes components of relatively varied chemical classes.

Source: The term “source” as used herein, typically refers to a context in which an agent of interest (e.g., that may be or comprise a carbohydrate, a lipid, a nucleic acid, a metal, polypeptide, a small molecule, or a combination thereof) may be found in nature, or from which such agent can be or has been obtained (e.g., isolated). In some embodiments, a source may be or comprise a biological source (e.g., an organism, tissue, or cell, or sample thereof); in some embodiments, a source may be an environmental source. In some embodiments, a source may be or comprise a primary sample from an organism (e.g., which may be or comprise a tissue or fluid of such organism, and/or may be or comprise cell(s) of such organism). In some embodiments, an organism may be or comprise a prokaryotic organism (e.g., a bacterium) or a eukaryotic organism (e.g., a fungus or yeast, an insect, a mammal, a plant, a reptile, etc.). In some embodiments, an infectious agent such as a virus or phage may be considered an organism for purposes of this disclosure, and in particular with respect to being a source. In some embodiments, a source may be or comprise an engineered source, such as a cell line or culture, an in vitro system, etc.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Target Locus: As used herein, the term “target locus” may refer to a specific site or location on a chromosome of interest. For example, a target locus may be a site to be “engineered” or “altered” by the hand of man. In some embodiments described and/or utilized herein, an engineered polynucleotide comprises homology to a target locus in order to allow further alterations at a specific site (e.g., CCR5 as a target locus, whose homologous sequence may be part of a guide RNA to result in incorporation of an edit via CRISPR/Cas-mediated gene editing). In some embodiments, target locus may interchangeably refer to a target gene of interest for manipulation by man. In some embodiments, such target locus manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols).

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more biological or chemical moieties as compared to the reference entity. In some embodiments, a variant also differs functionally from its reference molecule. In some embodiments, a variant differs structurally but performs the same or similar function as its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a variant polypeptide or nucleic acid is a truncated form of the reference polypeptide or nucleic acid. In some embodiments, a variant polypeptide that is a truncated form of the reference polypeptide may demonstrate comparable, identical, or greater levels of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into a viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into a genome of a host cell upon introduction into a host cell, and thereby are replicated along with a host genome. Moreover, certain vectors are capable of directing the expression of genes to which they are operatively linked. Such vectors are referred to herein as “expression vectors.”

### DETAILED DESCRIPTION OF CERTAIN EMBODIMENTS

### Cell therapy

Cell-based therapeutics (cell therapies) are an emerging class of medicine that make use of innate cellular machinery to combat disease. Unlike many traditional treatment methods, cell therapies make use of cellular localization, migration, and proliferation within the body, which can translate to improved biodistribution and targeted delivery of therapeutics. Cell therapies also benefit from cellular ability to sense and respond to various extrinsic signals within a subject, including, e.g., small molecules, other cells, physical forces, and/or marker proteins. Cellular persistence in vivo also enables cell therapies to survive, differentiate, function, etc. within a subject over extended time periods. These innate qualities may also lead to improved safety and efficacy for cell therapies as compared to other biologics or pharmaceutical compounds, providing long-lived, specifically targeted, adjustable, and/or responsive treatment for disease.

Cell-based therapies may have potential applications for a broad range of diseases, including those that have proved intractable or difficult to manage with traditional treatment options. Diseases that have been targeted for cell-based treatment include, e.g., various cancers, autoimmune diseases, central nervous system (CNS) diseases, neurodegenerative disorders, and cardiovascular diseases, among others. Cellular therapeutics offer an alternative over other treatment options for diseases where highly specific targeting (e.g., to a particular tissue type, area of the body, etc.) and/or longer-term treatment efficacy (e.g., enabling lower dosage frequency, single treatment options, etc.) are highly favored or necessary.

Cell therapies may employ a number of different cell types, which are typically modified to provide a therapeutic effect (e.g., transgene expression, reprogrammed cellular targeting, etc.). Although many cell types have potential to provide some form of therapeutic effect, recent therapies have heavily favored adaptive immune cells such as T lymphocytes and B lymphocytes (referred to interchangeably herein as T cells and B cells, respectively). For example, chimeric antigen receptor T (CAR-T) cells have been engineered to treat various cancers through recognition of one or more tumor cell markers, leading to cytotoxic destruction of tumor cells. CAR-T cells are also being adapted to treat infectious diseases (e.g., HIV) through recognition of other targeted antigens. Recent engineering efforts are focused on enhancing CAR-T receptor functionality, reducing innate immune response to CAR-T, and developing allogeneic therapies that make use of donor cells.

### B Lineage Cell Therapies

While cell-based therapies present an exciting new avenue for treatment of diseases, there are numerous challenges including achievement of safe, specific, and long-term therapeutic changes within targeted cells or tissues while reducing off-target effects. Furthermore, immune tolerance of these cell-based therapies is essential in order to obviate any deleterious side effects (See Jeske et al. 2021, incorporated by reference herein in its entirety). In order to address these challenges, engineering of B lineage cells have also been an area of development for cell-based therapies, due to the natural role of B lineage cells in antibody production within the body while minimizing inflammation. Antibody-based treatments are an established, well-studied form of treatment for a number of diseases, including cancer, autoimmune diseases, and infectious diseases. Monoclonal antibodies can be produced to target antigens within the body, which is valuable for treatment of diseases where such antibodies cannot be induced through natural processes (e.g., self-antigens for cancer and/or autoimmune diseases, antigens that fail to elicit natural immune response through infection and/or vaccination, etc.). Current antibody therapies require frequent administration and are costly to produce. Researchers have attempted to address these issues through use of gene therapy, which makes use of various techniques (e.g., viral vectors, CRISPR/Cas9 editing) to deliver antibody expression cassettes to endogenous cells in the body, leading to persistent antibody production within a subject. However, these approaches can result in low levels of antibody expression and a counter-active response by the subject's own immune system.

Furthermore, due to their minimal impact on a subject's native immune system and capacity to continually produce antibodies, B lineage cells are a highly desirable cell-based target to secrete other payloads including, but not limited to, enzymes, complement proteins, cytokines, cytokine receptors, chimeric antigen receptors (CARs), anti-fibrotic molecules, anti-thrombotic molecules, antigens, both wild type and variant proteins, coagulation factors, glucose response elements, and fragments of antibodies, antigens, and proteins. Recent reports have described successful engineering of B lineage cells, for example, producing human B-cell activating factor (hBAFF) in a murine model. Excitingly, this work not only demonstrates successful production of hBAFF, but also engraftment in the bone marrow and viability of these engineered B lineage cells up to sixty days post-engraftment (See, Cheng et al. 2022, incorporated herein by reference in its entirety). B lineage cells are an attractive option for development of cell-based therapies and may potentially offer improved therapeutic effects (e.g., payload delivery, targeting, long-term payload expression, reduced auto-immune response, etc.) as compared to traditional therapies (e.g., antibody-based therapeutics, other cell therapies, etc.).

In some embodiments, a B lineage cell is a cell that expresses one or more B cell receptors (BCR) on a cell membrane. In some embodiments, a B lineage cell is a modified version or variant of a cell that expresses one or more B cell receptors (BCRs) on a cell membrane. In some embodiments, a B lineage cell is a naïve or memory B cell. In some embodiments, a B lineage cell is a cell derived from a naïve B cell (e.g., activated B lineage cell, plasmablast, plasma cell) or a variant thereof. In some embodiments, a B lineage cell is an activated B lineage cell. In some embodiments, a B lineage cell is a plasmablast. In some embodiments, a B lineage cell is a plasma cell.

In some embodiments, a B lineage cell population comprise naïve B cells. In some embodiments, naïve B cell populations are used as reference cell populations. In some embodiments, naïve B cell populations express CD19 (CD19⁺). In some embodiments, expression of CD19 in naïve B cell populations is used as a reference to assist in characterization of other B lineage cell populations. In some embodiments, naïve B cell populations express CD20 (CD20⁺). In some embodiments, expression of CD20 in naïve B cell populations is used as a reference to assist in characterization of other B lineage cell populations. In some embodiments, naïve B cell populations express low amounts of CD27 (CD27^{lo}). In some embodiments, expression of CD27 in naïve B cell populations are as a reference to assist in characterization of other B lineage cell populations. In some embodiments, naïve B cell populations express low amounts of CD38 (CD38^{lo}). In some embodiments, expression of CD38 in naïve B cell populations are used as a reference to assist in characterization of other B lineage cell populations. In some embodiments, naïve B cell populations express low amounts of CD138 (CD138^{lo}). In some embodiments, expression of CD138 in naïve B cell populations are used as a reference to assist in characterization of other B lineage cell population.

In some embodiments, a B lineage cell population comprises activated B lineage cells. In some embodiments, activated B lineage cell populations are used as reference cell populations. In some embodiments, activated B lineage cell populations are compared to a reference cell population (e.g., naïve B cell population). In some embodiments, activated B lineage cell populations express lower amounts of CD19 (CD19^{lo}) as compared to a reference cell population (e.g., naïve B cell populations). In some embodiments, activated B lineage cell populations express different (e.g., higher or lower) amounts of CD19 as compared to a reference cell population (e.g., differentiated B cell populations, etc.). In some embodiments, activated B lineage cell populations express lower amounts of CD20 (CD20^{lo}) as compared to a reference cell population (e.g., naïve B cell populations). In some embodiments, activated B lineage cell populations express different (e.g., higher or lower) amounts of CD20 as compared to a reference cell population (e.g., differentiated B cell populations, etc.). In some embodiments, activated B lineage cell populations express higher amounts of CD27 (CD27^{hi}) as compared to a reference cell population (e.g., naïve B cell populations). In some embodiments, activated B lineage cell populations express different (e.g., higher or lower) amounts of CD27 as compared to a reference cell population (e.g., differentiated B cell populations, etc.). In some embodiments, activated B lineage cell populations express lower amounts of CD38 (CD38^{lo}) as compared to a reference cell population (e.g., naïve B cell populations). In some embodiments, activated B lineage cell populations express different (e.g., higher or lower) amounts of CD38 as compared to a reference cell population (e.g., differentiated B cell populations, etc.). In some embodiments, activated B lineage cell populations express lower amounts of CD138 (CD138^{lo}) as compared to a reference cell population (e.g., naïve B cell populations). In some embodiments, activated B lineage cell populations express different (e.g., higher or lower) amounts of CD138 as compared to a reference cell population (e.g., differentiated B cell populations, etc.).

In some embodiments, a B lineage cell population may comprise plasmablast cells. In some embodiments, plasmablast cell populations are used as reference cell populations. In some embodiments, plasmablast cell populations are compared to a reference cell population (e.g., naïve B cell population). In some embodiments, plasmablast cell populations express lower amounts of CD19 (CD19^{lo}) as compared to a reference cell population (e.g., naïve B cell populations). In some embodiments, plasmablast cell populations express different (e.g., higher or lower) amounts of CD19 as compared to a reference cell population (e.g., activated cell populations, plasma cell populations, etc.). In some embodiments, plasmablast cell populations express lower amounts of CD20 (CD20^{lo}) as compared to a reference cell population (e.g., naïve B cell populations). In some embodiments, plasmablast cell populations express different (e.g., higher or lower) amounts of CD20 as compared to a reference cell population (e.g., activated cell populations, plasma cell populations, etc.). In some embodiments, plasmablast cell populations express higher amounts of CD27 (CD27^{hi}) as compared to a reference cell population (e.g., naïve B cell populations). In some embodiments, plasmablast cell populations express different (e.g., higher or lower) amounts of CD27 as compared to a reference cell population (e.g., activated cell populations, plasma cell populations, etc.). In some embodiments, plasmablast cell populations express higher amounts of CD38 (CD38^{hi}) as compared to a reference cell population (e.g., naïve B cell populations). In some embodiments, plasmablast cell populations express different (e.g., higher or lower) amounts of CD38 as compared to a reference cell population (e.g., activated cell populations, plasma cell populations, etc.). In some embodiments, plasmablast cell populations express lower amounts of CD138 (CD138^{lo}) as compared to a reference cell population (e.g., naïve B cell populations). In some embodiments, plasmablast cell populations express different (e.g., higher or lower) amounts of CD138 as compared to a reference cell population (e.g., activated cell populations, plasma cell populations, etc.).

In some embodiments, a B lineage cell population comprises plasma cells. In some embodiments, plasma cell populations are used as reference cell populations. In some embodiments, plasma cell populations are compared to a reference cell population (e.g., naïve B cell population). In some embodiments, plasma cell populations express lower amounts of CD19 (CD19^{lo}) as compared to a reference cell population (e.g., naïve B cell populations). In some embodiments, plasma cell populations express different (e.g., higher or lower) amounts of CD19 as compared to a reference cell population (e.g., activated cell populations, plasmablast populations, etc.). In some embodiments, plasma cell populations express lower amounts of CD20 (CD20^{lo}) as compared to a reference cell population (e.g., naïve B cell populations). In some embodiments, plasma cell populations express different (e.g., higher or lower) amounts of CD20 as compared to a reference cell population (e.g., activated cell populations, plasmablast populations, etc.). In some embodiments, plasma cell populations express higher amounts of CD27 (CD27^{hi}) as compared to a reference cell population (e.g., naïve B cell populations). In some embodiments, plasma cell populations express different (e.g., higher or lower) amounts of CD27 as compared to a reference cell population (e.g., activated cell populations, plasmablast populations, etc.). In some embodiments, plasma cell populations express higher amounts of CD38 (CD38^{hi}) as compared to a reference cell population (e.g., naïve B cell populations). In some embodiments, plasma cell populations express different (e.g., higher or lower) amounts of CD38 as compared to a reference cell population (e.g., activated cell populations, plasmablast populations, etc.). In some embodiments, plasma cell populations express higher amounts of CD138 (CD138^{hi}) as compared to a reference cell population (e.g., naïve B cell populations). In some embodiments, plasma cell populations express different (e.g., higher or lower) amounts of CD138 as compared to a reference cell population (e.g., activated cell populations, plasmablast populations, etc.).

### Cell Engineering

Production of engineered cells for various uses including, e.g., cell therapies is an active area of development. Genomic and epigenomic modifications, synthetic biology, and application of biomaterials may be employed to generate engineered cells with desired properties for therapeutic applications. Selection of an appropriate method for generating engineered cells is typically dependent upon the desired output and effects of cell therapy and requires optimization for different cell types, transgenes of interest, etc. It is understood in the art that engineering methods that are effective for a particular cell type may be less effective (or not viable) for another cell type. Furthermore, engineering methods may vary depending upon whether a therapy requires cellular localization, expression of an endogenous or exogenous protein, removal of an endogenous protein, etc.

### Genome Editing

Genome editing tools can alter a cellular genome to produce a desired therapeutic effect, e.g., expression of a therapeutic protein. Targeted nucleases (e.g., Cas proteins, TALENs, ZFNs, etc.), viral vectors (e.g., AAV, lentiviral, adenoviral, etc.), recombinases (e.g., Cre recombinase, Flp recombinase, PhiC31 integrase, etc.), and other tools may be employed to provide genetic modifications. Gene editing efficiency may vary depending on, e.g., cell type, desired function, and ease of delivery. Accordingly, editing methods often require extensive optimization to provide engineered cells with intended functionality for therapeutic applications.

### B Lineage Cell Engineering

Various B lineage cell engineering techniques are described in the art, including, e.g., CRISPR/Cas9, AAV, lentiviral, and recombinase-based methods. These methods are generally employed to introduce a payload (e.g., expression cassette, transgene, etc.) into naïve B cells in order to express a protein of interest. A payload may be designed for episomal expression, integration into a specific target locus (e.g., endogenous target gene locus), or integration into a non-specific locus (e.g., endogenous random or non-target gene locus). Payloads may be designed such that an endogenous target gene locus continues to produce functional protein and/or fulfill its natural function (non-disruptive integration). Payloads may also be designed to intentionally disrupt an endogenous target gene locus to produce lowered or non-detectable levels of functional protein and/or some amount of non-functional protein.

Methods for integration of a payload (e.g., transgene) into an endogenous target locus may include site-specific cleavage with a targeted nuclease (e.g., Cas protein, including Cas9), followed by integration of a payload (e.g., transgene) through an endogenous repair pathway (e.g., homologous recombination, homology-directed repair, etc.).

In some embodiments, a method of B lineage cell engineering is or comprises administration of an ribonucleoprotein (RNP) to a cell population. In some embodiments, a method of B lineage cell engineering is or comprises administration of a composition comprising a Cas protein complexed with guide RNA (gRNA) to a cell population. In some embodiments, a method of B lineage cell engineering is or comprises administration of a composition comprising a Cas9/guide RNA complex a cell population. In some embodiments, a method of B lineage cell engineering is or comprises administration of a composition comprising a Cas9/guide RNA complex a cell population. In some embodiments, a method of B lineage cell engineering is or comprises administration of a composition comprising a payload (e.g., transgene) of interest to a cell population. In some embodiments, a method of B lineage cell engineering is or comprises administration of a composition comprising a payload (e.g., transgene) to a cell population through use of a viral vector. In some embodiments, a method of B lineage cell engineering is or comprises administration of a composition comprising a payload (e.g., transgene) encapsulated within an AAV capsid (e.g., AAV2, AAV3, AAV5, AAV6, AAV8, etc.) to a cell population. In some embodiments, a method of B lineage cell engineering is or comprises administration of a composition comprising a transgene encapsulated within an AAV capsid (e.g., AAV2, AAV3, AAV5, AAV6, AAV8, etc.) in combination with or in addition to administration of a composition comprising a Cas9/gRNA complex.

In some embodiments, a method of B lineage cell engineering comprises a step of electroporation to facilitate cellular uptake of one or more engineering components. In some embodiments, a method of B lineage cell engineering comprises a step of electroporation to facilitate cellular uptake of a Cas9/gRNA complex. In some embodiments, a method of B lineage cell engineering comprises a step of electroporation to facilitate cellular uptake of a Cas9/gRNA complex. In some embodiments, a method of B lineage cell engineering comprises a step of electroporation to facilitate cellular uptake of a Cas9/gRNA complex and a payload. In some embodiments, a method of B lineage cell engineering may comprise a step of electroporation to facilitate cellular uptake of a Cas9/gRNA complex and a payload (e.g., transgene) encapsulated in an AAV capsid (AAV2, AAV3, AAV5, AAV6, AAV8, etc.). In some embodiments, a method of B lineage cell engineering comprises a step of electroporation to facilitate cellular uptake of a Cas9/gRNA complex and a payload (e.g., transgene) encapsulated in an AAV6 capsid.

In some embodiments, a method of B lineage cell engineering comprises a step of viral transduction to facilitate cellular uptake of a payload (e.g., transgene). In some embodiments, a method of B lineage cell engineering comprises a step of viral transduction to facilitate cellular uptake of a payload (e.g., transgene) encapsulated in an AAV capsid (AAV2, AAV3, AAV5, AAV6, AAV8, etc.). In some embodiments, a method of B lineage cell engineering comprises a step of viral transduction to facilitate cellular uptake of a payload (e.g., transgene) encapsulated in an AAV6 capsid. In some embodiments, a method of B lineage cell engineering comprises one or more steps of: (i) electroporation to facilitate cellular uptake of a Cas9/gRNA complex; and (ii) viral transduction to facilitate cellular uptake of a payload encapsulated in an AAV capsid.

In some embodiments, a method of cell engineering is particularly effective for one type of cell (e.g., T cell) and less effective for another type of cell (e.g., B cell). In some embodiments, a method of B lineage cell engineering provides improved genome editing efficiency in B lineage cells as compared to other cell types (e.g., T cell). In some embodiments, a method of B cell engineering provides at least about 10%, 15%, 20%, 25%, 30%, 35%, 40%, 45%, 50%, 55%, 60%, 65%, 70%, 75%, 80%, 85%, 90%, 95%, or 99% editing efficiency. In some embodiments, a method of B lineage cell engineering provides at least about 1E6, 1.5E6, 2E6, 2.5E6, 3E6, 3.5E6, 4E6, 4.5E6, 5E6, 5.5E6, 6E6, 6.5E6, 7E6, 7.5E6, 8E6, 8.5E6, 9E6, 9.5E6, 1E7, 1.5E7, 2E7, 2.5E7, 3E7, 3.5E7, 4E7, 4.5E7, or 5E7 edited B lineage cells.

In some embodiments, a method of B lineage cell engineering comprises a step of editing activated B cells. In some embodiments, a method of B lineage cell engineering comprises a step of editing B lineage cells after an activation step of about 1, 2, 3, 4, or 5 day(s). In some embodiments, a method of B lineage cell engineering comprises a step of editing B lineage cells after an activation step of 2 days. In some embodiments, a method of B lineage cell engineering comprises a step of editing B lineage cells after an activation step of about 1, 2, 3, 4, or 5 day(s) and expanding edited B lineage cells in the activation media for an additional period of about 1, 2, 3, 4, 5, 6, 7, or 8 days. In some embodiments, a method of B lineage cell engineering comprises a step of editing B lineage cells after an activation step of 2 days and expanding edited B lineage cells in the activation media for an additional 6 days.

### Payloads

Various methods described herein may be used for generation of engineered cells (e.g., B lineage cell populations, etc.) comprising one or more payloads. In some embodiments, engineered cells (e.g., B lineage cell populations, etc.) comprise a polynucleotide sequence encoding one or more payloads. In accordance with various aspects of the present disclosure, any of a variety of payloads may be used (e.g., those with a therapeutic or monitoring purpose), alone or in combination. In some embodiments, a payload is or comprises a polynucleotide sequence encoding a peptide or polypeptide. In some embodiments, a payload is or comprises one or more transgenes. In some embodiments, a payload is or comprises one or more homology arm sequence. In some embodiments, a payload is or comprises a transgene flanked by one or more homology sequences.

### Transgenes

In some embodiments, a transgene is a corrective gene chosen to improve one or more signs and/or symptoms of a disease, disorder, or condition. In some embodiments, transgenes are functional versions of disease associated genes (i.e., gene isoform(s) which are associated with the manifestation or worsening of a disease, disorder or condition) found in a subject. In some embodiments, transgenes are an optimized version of disease-associated genes found in a subject (e.g., codon optimized or expression-optimized variants). In some embodiments, transgenes are variants of disease-associated genes found in a subject (e.g., functional gene fragment or variant thereof). In some embodiments, a transgene is a gene that causes expression of a peptide that is normally expressed in one or more healthy tissues.

In some embodiments, a transgene is a gene that causes expression of an altered protein with a gain- or loss-of-function mutation. In some embodiments, a transgene is a gene that causes expression of a fusion protein. In some embodiments, a transgene is a gene that causes expression of an antibody agent. In some embodiments, a transgene is a gene that causes expression of a multispecific antibody. In some embodiments, a transgene is a fragment of an antibody, antigen, or protein. In some embodiments, a transgene is a gene that causes expression an enzyme (e.g., for enzyme replacement therapy). In some embodiments, a transgene is a gene that causes expression of a cytokine. In some embodiments, a transgene is a gene that causes expression of a cytokine receptor. In some embodiments, a transgene is a gene that causes expression of a chimeric antigen receptor (CAR). In some embodiments, a transgene is a gene that causes expression of an anti-thrombotic molecule. In some embodiments, a transgene is a gene that causes expression of a coagulation factor. In some embodiments, a transgene is a gene that causes expression of a glucose response element. In some embodiments, a transgene is a gene that causes expression of a nanobody. In some embodiments, a transgene is a gene that causes expression of Factor IX (FIX) or a variant thereof. In some embodiments, a transgene is a gene that causes expression of sphingomyelin phosphodiesterase 1 (SMPD1) or a variant thereof.

In some embodiments, a transgene is or comprises a gene encoding a functional nucleic acid. In some embodiments, a therapeutic agent is or comprises an agent that has a therapeutic effect upon a host cell or subject (including, e.g., a ribozyme, guide RNA (gRNA), antisense oligonucleotide (ASO), miRNA, siRNA, and/or shRNA). For example, in some embodiments, a therapeutic agent promotes a biological process to treat a medical condition, e.g., at least one symptom of a disease, disorder, or condition.

In some embodiments, transgene expression in a subject results substantially from integration at a target locus. In some embodiments, 75% or more (e.g., 80% or more, 85% or more, 90% or more, 95% or more, 99% or more, 99.5% or more) of total transgene expression in a subject is from transgene integration at a target locus. In some embodiments, 25% or less (e.g., 20% or less, 15% or less, 10% or less, 5% or less, 1% or less, 0.5% or less, 0.1% or less) of total transgene expression in a subject is from a source other than transgene integration at a target locus (e.g., episomal expression, integration at a non-target locus).

In some embodiments, a transgene is or comprises a sequence having at least 80%, 85%, 90%, 95%, 99%, or 100% identity to a corresponding wild-type reference nucleotide sequence (e.g., a wild-type gene sequence). In some embodiments, a transgene is or comprises a sequence having a least 80%, 85%, 90%, 95%, 99%, or 100% identity to a portion of a corresponding wild-type reference nucleotide sequence (e.g., a wild-type gene sequence).

### Targeted Integration

In some embodiments, compositions disclosed herein direct integration of a payload (e.g., a transgene) at a target locus (e.g., an endogenous gene). In some embodiments, compositions and constructs provided herein direct integration of a payload at a target locus in a specific cell type (e.g., naïve B cells, B lineage cell populations, etc).

In some embodiments, compositions and constructs provided herein direct integration of a payload at a target locus that is considered a safe-harbor site (e.g., CCR5, AAVS1). In some embodiments, a target locus is selected from any genomic site appropriate for use with methods and compositions provided herein. In some embodiments, a target locus encodes a polypeptide. In some embodiments, a target locus encodes a polypeptide that is highly expressed in a subject (e.g., a subject not suffering from a disease, disorder, or condition, or a subject suffering from a disease, disorder, or condition). In some embodiments, a target locus is selected from one or more of CD19, CD20, IGH, B2M, CCR5, JCHAIN, PAX5, IRF4, IRF8, BACH2, EZH2, XBP1, CARD11, PRDM1, and BAFF.

### B Lineage Cell Culture Methods

Various methods for culturing and long-term maintenance of B lineage cells in vitro are described in the art (See, Rawlings et al. 1995, Rawlings et al 1997, and Fluckinger et al. 1998, each of which is incorporated by reference herein in its entirety). B lineage cell culturing conditions can significantly affect normal human B lineage development and for production of mature Ig-secreting B cells. Without wishing to be bound by any theory, it is generally thought that activation of B lineage cells ex vivo may be necessary for later genome editing approaches that make use of homology-directed repair (HDR), as required DNA repair proteins are present during the G₂/S phases of the cell cycle (See, Rogers and Cannon 2021, incorporated herein by reference in its entirety).

### Activation of B Lineage Cells

Various technologies for activation of B lineage cells in vitro have been described (also interchangeably referred to as “activated B lineage cells”). Traditionally, B lineage cell activation and proliferation in vitro employed CD40L-expressing feeder cell layer systems. Such feeder cell systems were described as difficult to standardize and often unreliable for providing consistent levels of activation and proliferation of B lineage cells. Recent advances have shifted to protocols for in vitro activation and proliferation of B lineage cells in specific culture systems comprising cytokines or other components in the absence of feeder cells (See Jourdan et al., 2009 and Hartweger et al., 2019, each of which is incorporated herein by reference in its entirety).

In some embodiments, methods for B lineage cell activation comprise contacting cells with media comprising one or more components of the present disclosure. In some embodiments, methods for B lineage cell activation comprise contacting cells with media comprising one or more cytokines and/or oligonucleotides (e.g., multimeric human CD40L, IL-2, IL-10, IL-15, IL-21 and/or CpG). In some embodiments, methods for B cell activation comprise contacting cells with media comprising at least about 5 ng/ml, 10 ng/mL, 15 ng/ml, 20 ng/ml, 25 ng/ml, 30 ng/ml, 35 ng/ml, 40 ng/ml, 45 ng/ml, 50 ng/ml, 55ng/ml, 60 ng/ml, 65 ng/ml, 70 ng/ml, 75 ng/ml, 80 ng/ml, 85 ng/ml, 90 ng/ml, 95 ng/ml, 100 ng/ml, 150 ng/ml, 200 ng/ml, or 500 ng/ml of one or more cytokines and/or oligonucleotides (e.g., multimeric human CD40L, IL-2, IL-10, IL-15, IL-21 and/or CpG). In some embodiments, methods for B lineage cell activation comprise contacting cells with media comprising at least about 0.1 ug/mL, 0.2 ug/mL, 0.3 ug/mL, 0.4 ug/mL, 0.5 ug/mL, 0.6 ug/mL, 0.7 ug/mL, 0.8 ug/mL, 0.9 ug/mL, 1 ug/mL, 1.5 ug/mL, 2 ug/mL, 2.5 ug/mL, 3 ug/mL, 3.5 ug/mL, 4 ug/mL, 4.5 ug/mL, or 5 ug/mL of one or more cytokines and/or oligonucleotides (e.g., multimeric human CD40L, IL-2, IL-10, IL-15, IL-21 and/or CpG).

In some embodiments, methods for B lineage cell activation comprise a step of contacting cells with media comprising one or more components of the present disclosure for at least about 1, 2, 3, 4, or 5 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least about 1, 2, 3, 4, or 5 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of gene editing. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of B lineage cell expansion. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of B lineage cell expansion for at least about 1, 2, 3, 4, 5, 6, 7, or 8 days.

In some embodiments, methods described herein result in an activated B lineage cell population.

### Differentiation of B Cells Into Plasmablasts

Plasmablasts are short-lived, rapidly produced effector cells that are primarily present in early antibody response and are one potential product of terminal B cell differentiation. Plasmablasts are capable of secreting antibodies, including IgM subtype antibodies, in order to mount an immediate response to certain antigens in the body.

Differentiation of B cells into plasmablasts in vitro may be promoted through use of certain signaling molecules, including one or more cytokines (e.g., IL-2, IL-6, IL-10, and/or IL-15). A variety of methods for differentiation of B cells into plasmablasts are known, but not limited to, those outlined in WO/2018/170150, incorporated herein by reference in its entirety. Plasmablasts produced by such methods may be characterized as cells that are CD27⁺/CD38⁺/CD138⁻.

In some embodiments, methods for B cell differentiation into plasmablasts comprise contacting cells with media comprising one or more components. In some embodiments, methods for B cell differentiation into plasmablasts comprise contacting activated B lineage cells with media comprising one or more cytokines (e.g., IL-2, IL-6, IL-10, and/or IL-15). In some embodiments, methods for B cell differentiation into plasmablasts comprise contacting activated B lineage cells with media comprising at least about 0.5 ng/ml, 1 ng/ml, 1.5 ng/ml, 2 ng/ml, 2.5 ng/ml, 3 ng/ml, 3.5 ng/ml, 4 ng/ml, 4.5 ng/ml, 5 ng/ml, 10 ng/ml, 15 ng/ml, 20 ng/ml, 25 ng/mL, 30 ng/ml, 35 ng/ml, 40 ng/ml, 45 ng/ml, 50 ng/ml, 55 ng/ml, 60 ng/ml, 65 ng/ml, 70 ng/ml, 75 ng/ml, 80 ng/ml, 85 ng/ml, 90 ng/ml, 95 ng/ml, or ng/ml of one or more cytokines (e.g., IL-2, IL-6, IL-10, and/or IL-15).

In some embodiments, methods for B cell differentiation into plasmablasts comprise a step of contacting cells with media comprising one or more components of the present disclosure for at least about 1, 2, 3, or 4 days. In some embodiments, methods for B cell differentiation into plasmablasts comprise a step of contacting activated B lineage cells with media comprising one or more cytokines (e.g., IL-2, IL-6, IL-10, and/or IL-15) for at least about 1, 2, 3, or 4 days. In some embodiments, methods for B cell differentiation into plasmablasts comprise a step of contacting activated B lineage cells with media comprising one or more cytokines (e.g., IL-2, IL-6, IL-10, and/or IL-15) for at least 3 days. In some embodiments, methods for B cell differentiation into plasmablasts comprise a step of contacting activated B lineage cells with media comprising one or more cytokines (e.g., IL-2, IL-6, IL-10, and/or IL-15) for at least 3 days, followed by a step of plasmablast expansion.

### Differentiation of Plasmablasts Into Plasma Cells

Although plasmablasts secrete more antibodies than naïve B cells, they are shorter-lived and secrete fewer antibodies than plasma cells (PCs). Long-lived plasma cells (LLPCs, used interchangeably throughout with plasma cells) localize to bone marrow in the body and are capable of secreting high levels of antibodies and surviving for decades in the absence of proliferation (See, Hammerland et al., 2017 and Khodadadi et al., 2019, both of which incorporated herewith in their entirety). Differentiation of plasmablasts to long-lived plasma cells can be triggered by certain events in the body, including, e.g., activity of transcription factors Blimp-1/PRDM1 and IRF4. Differentiation of plasmablasts to plasma cells in vitro may be promoted through use of certain signaling molecules, including one or more cytokines (e.g., IL-6, IL-15, and/or IFNα-2β). A variety of methods for differentiation of plasmablasts into plasma cells are known, but not limited to, those outlined in Jourdan et al. 2019 and WO/2018/170150 (each of which incorporated herein by reference in its entirety). Plasma cells produced by such methods may be characterized as cells that are CD27⁺/CD38⁺/CD138⁺.

In some embodiments, methods for plasmablast differentiation into plasma cells comprise contacting cells with media comprising one or more cytokines (e.g., IL-6, IL-15, and/or IFNα-2β). In some embodiments, methods for plasmablast differentiation into plasma cells comprise contacting plasmablasts with media comprising one or more cytokines (e.g., IL-6, IL-15, and/or IFNα-2β). In some embodiments, methods for plasmablast differentiation into plasma cells comprise contacting plasmablasts with media comprising at least about 0.5 ng/ml, 1 ng/ml, 1.5 ng/ml, 2 ng/ml, 2.5 ng/ml, 3 ng/ml, 3.5 ng/ml, 4 ng/ml, 4.5 ng/ml, 5 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/mL, 60 ng/ml, 65 ng/ml, 70 ng/ml, 75 ng/ml, 80 ng/ml, 85 ng/ml, 90 ng/ml, 95 ng/ml, or ng/ml of one or more cytokines (e.g., IL-6, IL-15, and/or IFNα-2β).

In some embodiments, methods for plasmablast differentiation into plasma cells comprise a step of contacting cells with media comprising one or more components of the present disclosure for at least about 1, 2, 3, or 4 days. In some embodiments, methods for plasmablast differentiation into plasma cells comprise a step of contacting plasmablasts with media comprising one or more cytokines (e.g., IL-6, IL-15, and/or IFNα-2β) for at least about 1, 2, 3, or 4 days. In some embodiments, methods for plasmablast differentiation into plasma cells comprise a step of contacting plasmablasts with media comprising one or more cytokines (e.g., IL-6, IL-15, and/or IFNα-2β) for at least 3 days. In some embodiments, methods for plasmablast differentiation into plasma cells comprise a step of contacting plasmablasts with media comprising one or more cytokines (e.g., IL-6, IL-15, and/or IFNα-2β) for at least 3 days, followed by a step of cell isolation. In some embodiments, methods for plasmablast differentiation into plasma cells comprise a step of contacting plasmablasts with media comprising one or more cytokines (e.g., IL-6, IL-15, and/or IFNα-2β) for at least 3 days, followed by a step of administration to a subject.

### Cryopreservation of Cell Populations

Cell therapies provide a number of potential advantages over traditional treatment options, at least in part due to the unique properties of viable cells within the body. However, it has been recognized by those in the art that successful clinical outcomes require that cell therapies be quickly and effectively delivered to a subject. Thus, it is essential to develop preservation and storage techniques to maintain a standardized efficacy until they can be transported and administered to a subject. Furthermore, it is equally crucial to recognize and identify conditions that will increase viability and function of the cell population, as each cell population has different structural considerations for preservation (See, Meneghel, 2020, incorporated herein by reference in its entirety). Numerous people have worked hard to try to address these concerns and develop useful preservation technologies, but these again are very cell-specific and can lead to various inadequacies. For example, there are reports describing significant drops in B lineage cell subpopulations when attempting to preserve over an extended duration (See, Ticha et al., 2021, incorporated herein by reference in its entirety). The present disclosure seeks to address these challenges in the field by providing novel culturing conditions, methodology and technologies that achieve effective cryopreservation of certain cell populations (e.g., engineered B lineage cell populations).

Storage conditions are one of several considerations necessitated for cryopreservation. Short-term storage at lowered temperatures (e.g., 2-4 days at 4° C.) may not sufficiently preserve cell therapies such that they maintain normal metabolic processes, viability, and/or expression of any payload of interest. Accordingly, cell therapies often need stable storage at temperatures below −130° C., which prevents cellular metabolic changes. Cryopreservation processes are well-established for certain cell types, such as hematopoietic stem cells (HSCs) for transplantation, with over 47,000 procedures carried out in Europe in 2018 (See, Passweg et al, 2020, incorporated herein by reference in its entirety). In order to maintain consistency and reproducibility of cell therapies, cryopreservation protocols must be clear and precise, with specific requirements for each step of freezing, storage, and thawing prior to patient administration. Cryopreservation may also be used at multiple points in a process chain for cell therapy, e.g. multiple times between isolation of cells and patient administration.

Although various compositions and components for cryopreservation have been tested for a number of different cell types, optimal cryopreservation conditions may vary between cell therapies depending on specific requirements and features of each therapy. For example, in some embodiments, cryopreservation conditions that preserve activity of one type of cell therapy do not maintain activity of another type of cell therapy. Similarly, in some embodiments, cryopreservation conditions that preserve viability of a particular cell type do not maintain viability and/or activity of a cell therapy employing an engineered cell of the same cell type.

The present disclosure provides certain technologies that achieve effective cryopreservation of certain cell populations (e.g., of certain B lineage cell populations).

### Cryoprotective Agents (CPAs)

Cryopreservation may cause a number of cellular injuries, including, e.g., potentially harmful changes in cell morphology, characteristics (e.g., adhesion, cell surface markers, protein expression profile), metabolic activity (e.g., proliferative ability, potency), and/or function (e.g., immunomodulation, signal responsiveness), and may also lead to cell death. Temperature changes associated with cryopreservation methods may cause an adverse response in cells, including, e.g., activation of certain stress response pathways upon initial freezing and/or activation of apoptotic and necrotic pathways upon thawing. Ice crystal formation in media during cryopreservative freezing can also cause significant cellular damage through, e.g., physical interruption of cellular functions and processes and through solute exclusion and increased localized solute concentration, which may result in increased osmolarity and osmotic stress (See, Meneghel et al, 2020). Temperature changes may also lead to intracellular ice formation, which can cause cell damage and death.

In order to mitigate cellular injuries as a result of temperature changes, additive compounds known as cryoprotective agents (also referred to interchangeably herein as cryoprotectants) may be added to cell media. Cryoprotective agents (CPAs) have relatively low toxicity to cells, particularly at lower temperatures. CPAs may also exclude ice crystals and maintain a larger, unfrozen extracellular portion of media at certain temperatures, reducing osmotic effects and stress on cells (See, Meneghel et al, 2020). CPAs (e.g., DMSO) may also promote cell dehydration, limiting probability of intracellular ice formation and interacting with/stabilizing cellular membranes (See, Gao et al, 2000). CPAs that diffuse through the outer cell membrane may also be capable of altering intracellular osmotic concentration, reducing and/or regulating intracellular ice formation, dehydration, etc (See, Whaley et al 2021). Mixtures of permeable and non-permeable CPAs may be employed to provide desired effects and can be optimized for particular cell types (e.g., cell therapies) (See, Balci and Can 2013). Certain CPAs may also be optimized for exposure time, exposure temperature, loading time, loading temperature, etc., to balance cryoprotective effects with potential toxicity to cells. For example, permeable CPAs (e.g., DMSO) may have a cell membrane permeability that is approximately 100-1000 times lower than that of water, requiring a minimum period of incubation with cells in order to allow equilibration of protectant (See, Mazur et al, 2004).

In some embodiments, cryopreserved cell preparations provided and/or utilized in accordance with the present disclosure (e.g., cryopreserved B lineage cell preparation) comprise one or more CPAs disclosed herein. In some embodiments, cryopreserved cell preparations comprise one or more permeable CPAs. In some embodiments, cryopreserved cell preparations comprise DMSO. In some embodiments, cryopreserved cell preparations comprise glycerol. In some embodiments, cryopreserved cell preparations comprise ethylene glycol. In some embodiments, cryopreserved cell preparations comprise propylene glycol.

In some embodiments, cryopreserved cell preparations comprise one or more non-permeable CPAs. In some embodiments, cryopreserved cell preparations comprise glucose. In some embodiments, cryopreserved cell preparations comprise sucrose. In some embodiments, cryopreserved cell preparations comprise trehalose. In some embodiments, cryopreserved cell preparations comprise raffinose. In some embodiments, cryopreserved cell preparations comprise hydroxyethyl starch (HES). In some embodiments, cryopreserved cell preparations comprise polyvinylpyrrolidone (PVP). In some embodiments, cryopreserved cell preparations comprise polyvinyl alcohol (PVA). In some embodiments, cryopreserved cell preparations comprise polyethylene glycol (PEG). In some embodiments, cryopreserved cell preparations comprise dextran. In some embodiments, cryopreserved cell preparations comprise one or more albumin proteins. In some embodiments, cryopreserved cell preparations comprise human serum albumin (HSA). In some embodiments, cryopreserved cell preparations comprise blood serum. In some embodiments, cryopreserved cell preparations comprise poloxamers (e.g., P188). In some embodiments, cryopreserved cell preparations comprise mannitol. In some embodiments, cryopreserved cell preparations comprise glutathione. In some embodiments, cryopreserved cell preparations comprise thioredoxin. In some embodiments, cryopreserved cell preparations comprise glutathione. In some embodiments, cryopreserved cell preparations comprise ascorbic acid. In some embodiments, cryopreserved cell preparations comprise mitoquinone. In some embodiments, cryopreserved cell preparations comprise salidroside. In some embodiments, cryopreserved cell preparations comprise reservatrol. In some embodiments, cryopreserved cell preparations comprise N-acetyl-L-cysteine. In some embodiments, cryopreserved cell preparations comprise catalase. In some embodiments, cryopreserved cell preparations comprise α-tocopheryl acetate. In some embodiments, cryopreserved cell preparations comprise lactated ringers. In some embodiments, cryopreserved cell preparations comprise dextrose.

In some embodiments, cryopreserved cell preparations comprise one or more permeable CPAs and one or more non-permeable CPAs. In some embodiments, cryopreserved cell preparations comprise DMSO and HSA. In some embodiments, cryopreserved cell preparations comprise lactated ringers and dextrose.

In some embodiments, cryoprotectants are added to cell preparations in a particular order to facilitate cryopreservation (e.g., HSA followed by the addition of DMSO). In some embodiments, one or more non-permeable CPAs is added first, followed by a permeable CPA. In some embodiments, one or more non-permeable CPAs is added first, followed by another non-permeable CPA. In some embodiments, one or more permeable CPAs is added first, followed by a non-permeable CPA. In some embodiments, one or more permeable CPAs is added first, followed by another permeable CPA.

In some embodiments, cryoprotectants are added to cell preparations at a particular temperature in order to facilitate cryopreservation. In some embodiments, cryoprotectants are added at a temperature of about −5° C., −4° C., −3° C., −2° C., −1° C., 0° C., 1° C., 2° C., 3° C., 4° C., 5° C., 6° C., 7° C., 8° C., 9 ° C., or 10° C. In some embodiments, cryoprotectants are added at a temperature of about 4° C. In some embodiments, cryoprotectants are added at a temperature of about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C. In some embodiments, cryoprotectants are added at a temperature of about 20° C. In some embodiments, cryoprotectants are added at a temperature of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C. In some embodiments, cryoprotectants are added at a temperature of about 37° C.

In some embodiments, cryoprotectants are added to cell preparations and allowed to equilibrate during an incubation period to facilitate cryopreservation. In some embodiments, cell preparations are incubated with cryoprotectants at a temperature of about 5° C., −4° C., −3° C., −2° C., −1° C., 0° C., 1° C., 2° C., 3° C., 4° C., 5° C., 6° C., 7° C., 8° C., 9° C., or 10° C. In some embodiments, cell preparations are incubated with cryoprotectants at a temperature of about 4° C. In some embodiments, cell preparations are incubated with cryoprotectants at a temperature of about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C. In some embodiments, cell preparations are incubated with cryoprotectants at a temperature of about 20° C. In some embodiments, cell preparations are incubated with cryoprotectants at a temperature of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C. In some embodiments, cell preparations are incubated with cryoprotectants at a temperature of about 37° C.

In some embodiments, cell preparations are incubated with cryoprotectants for at least about 1, 2, 3, 4, 5, 6, 7, 8, 9, or 10 minutes. In some embodiments, cell preparations are incubated with cryoprotectants for at least about 10 minutes. In some embodiments, cell preparations are incubated with cryoprotectants for at least about 30, 31, 32, 33, 34, 35, or 36 minutes. In some embodiments, cell preparations are incubated with cryoprotectants for at least about at least about 36 minutes. In some embodiments, cell preparations are incubated with cryoprotectants for at least about 120, 121, 122, 123, 124, 125, 126, or 127 minutes. In some embodiments, cell preparations are incubated with cryoprotectants for at least about 127 minutes.

In some embodiments, cell preparations are exposed to cryoprotectants for a certain fixed time period prior to a freezing process. In some embodiments, cell preparations are exposed to cryoprotectants for no more than about 20, 21, 22, 23, 24, or 25 minutes. In some embodiments, cell preparations are exposed to cryoprotectants for no more than about 25 minutes. In some embodiments, cell preparations are exposed to cryoprotectants for no more than about 45, 46, 47, 48, 49, 50, or 51 minutes. In some embodiments, cell preparations are exposed to cryoprotectants for no more than about 51 minutes. In some embodiments, cell preparations are exposed to cryoprotectants for no more than about 130, 131, 132, 133, 134, 135, 136, 137, 138, 139, 140, 141, or 142 minutes. In some embodiments, cell preparations are exposed to cryoprotectants for no more than about 142 minutes.

### Freezing Methods

Cooling rate is another factor for consideration when optimizing cryopreservation protocols. Appropriate freezing rates may vary between cell types and can also be affected by media components and cryoprotectants. Cellular injury during freezing may arise from both extracellular and intracellular ice formation and controlled cooling rate can affect when and where ice forms (See, Meneghel et al 2020). Relatively slow cooling rates can result in ice forming first within an extracellular media, excluding solutes from the ice crystals and leading to increased osmolarity and potential osmotic shock to the cells. These changes in osmolarity can also lead to partial dehydration of cells, which can provide a beneficial effect by reducing probability of intracellular ice formation and/or stabilizing cell membranes. Relatively high cooling rates may prevent cellular dehydration from occurring prior to freezing, leading to increased intracellular ice levels and cellular damage. The present disclosure, among other things, recognizes that differences exist between various cell types in viability and function under different cooling rates. In order to address these cell-to-cell variations for cryopreservation, the present disclosure employs a process comprising of both heating and cooling phases in a process known as controlled cooling.

In some embodiments, a controlled cooling method can be used. In some embodiments, controlled cooling comprises a step of cooling a chamber until a cell preparation reaches about ±1-10° C. In some embodiments, controlled cooling comprises a step of cooling the chamber at a rate of ±1-25° C./minute until the cryopreservation cell preparation reaches about ±1-20° C. In some embodiments, controlled cooling comprises a step of cooling the chamber at a rate of ±1-25° C./minute until the chamber reaches about ±1-40° C. In some embodiments, controlled cooling comprises a step of heating the chamber at a rate of about ±1-10° C./minute until the chamber reaches about ±1-12° C. In some embodiments, controlled cooling comprises a step of cooling the chamber at a rate of ±1-10° C./minute until the chamber reaches ±1-40° C. In some embodiments, controlled cooling comprises a step of cooling the chamber at a rate of ±1-10° C./minute until the chamber reaches ±1-90° C.

In some embodiments, a controlled cooling method can be used. In some embodiments, controlled cooling comprises a step of cooling a chamber until a cell preparation reaches 4° C. In some embodiments, controlled cooling comprises a step of cooling the chamber at a rate of 1° C./minute until the cryopreservation cell preparation reaches −4° C. In some embodiments, controlled cooling comprises a step of cooling the chamber at a rate of 25° C./minute until the chamber reaches −40° C. In some embodiments, controlled cooling comprises a step of heating the chamber at a rate of 10° C./minute until the chamber reaches −12° C. In some embodiments, controlled cooling comprises a step of cooling the chamber at a rate of 1° C./minute until the chamber reaches −40° C. In some embodiments, controlled cooling comprises a step of cooling the chamber at a rate of 10° C./minute until the chamber reaches −90° C.

### Storage Methods

It is recognized by those in the art that large volumes of cells and tissues upon cryopreservation can be stored until the appropriate time and place for pre-clinical and clinical testing and delivery (See, Meneghel et al. 2020). Furthermore, cryopreservation and storage methods described herein provides numerous advantages over conventional short-term storage, including but not limited to, availability of preparations of consistent viability and functionality, flexibility and predictability for delivery, less cellular waste, reproducibility, and minimization of contamination.

In some embodiments, B lineage cell preparations may be stored in sterile cryovials (e.g., 1-50 ml). In some embodiments, such cryovials may be hermetically sealed. In some embodiments, B lineage cell preparations may be stored in a cryobag. In some embodiments, such cryobags may be hermetically sealed. In some embodiments, cryobags may additionally be stored within an overwrap bag. In some embodiments, cryobags may be stored in a metallic casing. In some embodiments, cryobags stored within an overwrap bag may be additionally stored in a metallic casing. In some embodiments, B lineage cell preparations may be stored in syringes.

In some embodiments, B lineage cell preparations are stored at −170° C. In some embodiments, B lineage cell preparations are stored in ultra-cold freezers (e.g., vapor-based liquid nitrogen-based freezer). In some embodiments, B lineage cell preparations are stored in liquid nitrogen-based containers. In some embodiments, B lineage cell preparations are stored in vapor phase above liquid nitrogen. In some embodiments, B lineage cell preparations are stored in cryogenic storage.

In some embodiments, B lineage cell preparations are transported through one or more methods to preserve a temperature of about −170° C. In some embodiments, B lineage cell preparations are transported in vapor phase above liquid nitrogen. In some embodiments, B lineage cell preparations are transported on dry ice.

### Thawing Methods

The present disclosure, among other things, describes cryopreserved engineered B lineage cell populations that can be used in both pre-clinical and clinical settings. Appropriate thawing rate must be considered in order to both successfully cryopreserved a cell population as well as for administration of said cell population. Thawing rate goes hand-in-hand with controlled cooling rate, as these are co-dependent processes. Rapid cooling will result in the amount of ice within the cryopreserved product to be lower than anticipated. As a circumstance, a relatively rapid rate of warming during thawing is required in order to obviate any potential cellular injuries to occur via further ice crystallization. Furthermore, this will minimize the level of hypertonic stress that such cryopreserved cell populations experience upon thawing and will result in optimal viability (See, Meneghel et al. 2020). It is recognized that slower controlled cooling rates, e.g., in cryobags, the proportion of extracellular ice will be greater. Thus, slower rates of warming during thawing would be necessary to reduced risk of ice recrystallization and reduced osmotic stress.

In some embodiments, the cryopreserved B lineage cell preparation is transferred from a cryogenic storage into a thawing mechanism. In some embodiments, a thawing mechanism is a dry heater. In some embodiments, a thawing mechanism is a water bath.

### Compositions

### Engineered Cell Preparations

The present disclosure describes engineered cell preparations comprising populations of cells modified to perform one or more desired functions. In some embodiments, engineered cell preparations are compositions comprising genetically modified immune cell populations (e.g., B cell, T cell). In some embodiments, engineered cell preparations are compositions comprising genetically modified B lineage cell populations (e.g., B cell, plasmablast, plasma cell). In some embodiments, engineered cell preparations are compositions comprising genetically modified plasmablast cell populations. In some embodiments, engineered cell preparations are compositions comprising genetically modified plasma cell populations.

In some embodiments, engineered cell preparations comprise genetically modified cells that express a payload (e.g., transgene) of interest. In some embodiments, engineered cell preparations comprise genetically modified cells that express a transgene of interest (e.g., therapeutic protein, antibody, etc.). In some embodiments, engineered cell preparations comprise genetically modified cells that express a transgene of interest (e.g., therapeutic protein, antibody, etc.) from an endogenous gene locus. In some embodiments, engineered cell preparations comprise genetically modified cells that express a transgene of interest (e.g., therapeutic protein, antibody, etc.) from an endogenous gene locus under control of an endogenous promoter. In some embodiments, engineered cell preparations comprise genetically modified cells that express a transgene of interest (e.g., therapeutic protein, antibody, etc.) from an endogenous gene locus under control of an exogenous promoter. In some embodiments, engineered cell preparations comprise genetically modified cells that express a transgene of interest (e.g., therapeutic protein, antibody, etc.) from an endogenous gene locus without disrupting endogenous gene expression and/or function. In some embodiments, engineered cell preparations comprise genetically modified cells that express a transgene of interest (e.g., therapeutic protein, antibody, etc.) from an endogenous gene locus and partially or fully disrupt endogenous gene expression and/or function.

### Fresh Engineered Cell Preparations

The present disclosure provides a number of different cell preparations that are freshly engineered (interchangeably referred to herein as “fresh engineered cell preparations”). In some embodiments, fresh engineered cell preparations are compositions comprising genetically modified immune cell populations (e.g., T cell, B cell, plasmablast, plasma cell). In some embodiments, fresh engineered cell preparations are compositions comprising genetically modified B lineage cell populations (e.g., B cell, plasmablast and plasma cell). In some embodiments, fresh engineered cell preparations are compositions comprising genetically modified plasmablast cell populations. In some embodiments, fresh engineered cell preparations are compositions comprising genetically modified plasma cell populations.

In some embodiments, fresh engineered cell preparations comprise genetically modified cells that express a payload (e.g., transgene) of interest. In some embodiments, fresh engineered cell preparations comprise genetically modified cells that express a transgene of interest (e.g., therapeutic protein, antibody, etc.). In some embodiments, fresh engineered cell preparations comprise genetically modified cells that express a transgene of interest (e.g., therapeutic protein, antibody, etc.) from an endogenous gene locus. In some embodiments, fresh engineered cell preparations comprise genetically modified cells that express a transgene of interest (e.g., therapeutic protein, antibody, etc.) from an endogenous gene locus under control of an endogenous promoter. In some embodiments, fresh engineered cell preparations comprise genetically modified cells that express a transgene of interest (e.g., therapeutic protein, antibody, etc.) from an endogenous gene locus under control of an exogenous promoter. In some embodiments, fresh engineered cell preparations comprise genetically modified cells that express a transgene of interest (e.g., therapeutic protein, antibody, etc.) from an endogenous gene locus without disrupting endogenous gene expression and/or function. In some embodiments, fresh engineered cell preparations comprise genetically modified cells that express a transgene of interest (e.g., therapeutic protein, antibody, etc.) from an endogenous gene locus and partially or fully disrupt endogenous gene expression and/or function.

The present disclosure provides methods and compositions for cryopreservation of fresh engineered cell preparations as described herein.

### Previously Frozen Engineered Cell Preparations

The present disclosure provides a number of different cell preparations that were previously engineered and frozen. In some embodiments, previously frozen engineered cell preparations are used as a reference point for cryopreserved cell preparations. In some embodiments, previously frozen engineered cell preparations are compositions comprising immune cell populations (e.g., T cell, B cell, plasmablast, and plasma cell). In some embodiments, previously frozen engineered cell preparations are genetically modified immune cell populations. In some embodiments, previously frozen engineered cell preparations are compositions comprising genetically modified B lineage cells (e.g., B cell, plasmablast, and plasma cell). In some embodiments, previously frozen engineered cell preparations are compositions comprising genetically modified plasmablast cell populations. In some embodiments, previously frozen engineered cell preparations are compositions comprising genetically modified plasma cell populations.

The present disclosure provides methods for thawing of previously frozen engineered cell preparations as described herein.

### Cryopreserved Engineered Cell Preparations

The present disclosure provides a number of different cell preparations that have undergone engineering and subsequent cryopreservation. In some embodiments, engineered cell preparations have undergone cryopreservation methods described herein. In some embodiments, cryopreserved engineered cell preparations are compositions comprising immune cell populations (e.g., T cell, B cell, plasmablast, plasma cell). In some embodiments, cryopreserved engineered cell preparations are genetically modified immune cell populations. In some embodiments, cryopreserved engineered cell preparations are compositions comprising genetically modified B lineage cell populations. In some embodiments, cryopreserved engineered cell preparations are compositions comprising genetically modified plasmablast cell populations. In some embodiments, cryopreserved engineered cell preparations are compositions comprising genetically modified plasma cell populations.

In some embodiments, cryopreserved engineered cell preparations comprise genetically modified cells that express a payload (e.g., transgene) of interest. In some embodiments, cryopreserved engineered cell preparations comprise genetically modified cells that express a transgene of interest (e.g., therapeutic protein, antibody, etc.). In some embodiments, cryopreserved engineered cell preparations comprise genetically modified cells that express a transgene of interest (e.g., therapeutic protein, antibody, etc.) from an endogenous gene locus. In some embodiments, cryopreserved engineered cell preparations comprise genetically modified cells that express a transgene of interest (e.g., therapeutic protein, antibody, etc.) from an endogenous gene locus under control of an endogenous promoter. In some embodiments, cryopreserved engineered cell preparations comprise genetically modified cells that express a transgene of interest (e.g., therapeutic protein, antibody, etc.) from an endogenous gene locus under control of an exogenous promoter. In some embodiments, cryopreserved engineered cell preparations comprise genetically modified cells that express a transgene of interest (e.g., therapeutic protein, antibody, etc.) from an endogenous gene locus without disrupting endogenous gene expression and/or function. In some embodiments, cryopreserved engineered cell preparations comprise genetically modified cells that express a transgene of interest (e.g., therapeutic protein, antibody, etc.) from an endogenous gene locus and partially or fully disrupt endogenous gene expression and/or function.

In some embodiments, cryopreserved engineered cell preparations comprise cell populations that have been previously frozen at −170° C. in media comprising one or more cryopreservation agent (e.g., HSA and DMSO). In some embodiments, cryopreserved engineered cell preparations comprise cell populations that have been previously frozen at −170° C. through one or more steps of controlled temperature changes in media comprising one or more of HSA, DMSO, plasmalyte, and CryoStor 10 (CS10). In some embodiments, media comprises HSA, DMSO, plasmalyte, and CryoStor 10 (CS10).

The present disclosure provides methods and compositions for cryopreservation and thawing of cryopreserved engineered cell preparation as described herein.

### Production

The present disclosure describes production of certain cryopreserved engineered B lineage cell preparations. As disclosed herein, in some embodiments, a cryopreserved B lineage cell preparation comprises both engineered and non-engineered cells. In some embodiments, a cryopreserved engineered B lineage cell population comprises plasmablasts. In some embodiments, a cryopreserved engineered B lineage cell population comprises plasma cells. In some embodiments, a cryopreserved engineered B lineage cell population comprises long-lived plasma cells. In some embodiments, a cryopreserved engineered B lineage cell population comprises plasmablasts, plasma cells, and/or long-lived plasma cells and/or any mixtures or combinations thereof disclosed herein.

### Plasmablast Preparation

In some embodiments, a cryopreserved engineered B lineage cell preparation comprises plasmablasts. As understood in the art, plasmablasts are rapidly produced and short-lived effector cells of the early antibody response. Plasmablasts can be generated from activated B lineage cells using methods described herein. In some embodiments, engineered plasmablasts may be generated from engineered activated B lineage cells.

In some embodiments, plasmablast cell populations may be contacted with media comprising one or more components of the present disclosure (e.g., IL-6, IL-15, and/or IFNα-2β) in order to initiate differentiation into plasma cell populations. In some embodiments, methods for plasmablast cell population differentiation into a plasma cell population comprise contacting plasmablast cell population with media comprising one or more cytokines (e.g., IL-6, IL-15, and/or IFNα-2β). In some embodiments, methods for a plasmablast cell population differentiation into a plasma cell population comprise contacting plasmablast cell population with media comprising at least about 0.5 ng/ml, 1 ng/ml, 1.5 ng/ml, 2 ng/ml, 2.5 ng/ml, 3 ng/ml, 3.5 ng/ml, 4 ng/ml, 4.5 ng/ml, 5 ng/ml, 10 ng/mL, 15 ng/mL, 20 ng/ml, 25 ng/ml, 30 ng/ml, 35 ng/ml, 40 ng/ml, 45 ng/ml, 50 ng/ml, 55 ng/ml, 60 ng/ml, 65 ng/ml, 70 ng/ml, 75 ng/ml, 80 ng/ml, 85 ng/ml, 90 ng/ml, 95 ng/ml, or ng/ml of one or more cytokines (e.g., IL-6, IL-15, and/or IFNα-2β).

In some embodiments, methods for plasmablast cell population differentiation into a plasma cell population comprise a step of contacting cells with media comprising one or more components of the present disclosure for at least about 1, 2, 3, or 4 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least about 1, 2, 3, or 4 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least 3 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least 3 days, followed by a step of cell isolation. In some embodiments, methods for plasmablast cell population differentiation into a plasma cell population comprise a step of contacting plasmablasts with media comprising one or more cytokines (e.g., IL-6, IL-15, and/or IFNα-2β) for at least 3 days, followed by a step of administration to a subject.

### Plasma Cell Preparation

In some embodiments, a cryopreserved engineered B lineage cell preparation comprises a plasma cell population. In some embodiments, a plasma cell population comprises a predetermined engineered plasma cell precursors (e.g., plasma blasts) that, upon administration to a subject, further differentiates into a mature plasma cell population. As understood in the art, a mature plasma cell population comprises quiescent, non-dividing cells of the humoral immune response that are capable of secreting large amounts of antibodies. In some embodiments, a mature plasma cell population can comprise short-lived plasma cells and/or long-lived plasma cells (LLPCs) and/or any combination thereof.

### Characterization

The present disclosure provides various methods for characterization of engineered cell populations (e.g., B lineage cell populations). In some embodiments, methods disclosed herein are used to segregate and/or characterize cryopreserved naïve B cell subpopulations within a cryopreserved B lineage cell population. In some embodiments, naïve B cell subpopulations are characterized through one or more of flow cytometry, fluorescence-activated cell sorting (FACS), magnetic-activated cell sorting (MACS), and/or affinity chromatography. In some embodiments, one or more methods of characterizing naïve B cell subpopulations are employed during each step of a controlled cooling method. In some embodiments, methods of characterization are employed to segregate and/or characterize engineered naïve B cell subpopulations. In some embodiments, engineered naïve B cell subpopulations are characterized through one or more of flow cytometry, fluorescence-activated cell sorting (FACS), magnetic-activated cell sorting (MACS), and/or affinity chromatography. In some embodiments, one or more methods of characterizing engineered naïve B cell subpopulations are employed during each step of a controlled cooling method.

In some embodiments, methods disclosed herein are used to segregate and/or characterize cryopreserved activated B lineage cell subpopulations within a cryopreserved B lineage cell population. In some embodiments, activated B lineage cell subpopulations are characterized through one or more of flow cytometry, fluorescence-activated cell sorting (FACS), magnetic-activated cell sorting (MACS), and/or affinity chromatography. In some embodiments, one or more methods of characterizing activated B lineage cell subpopulations are employed during each step of a controlled cooling method. In some embodiments, methods disclosed herein are used to segregate and/or characterize cryopreserved engineered activated B lineage cell subpopulations. In some embodiments, engineered activated B lineage cell subpopulations are characterized through one or more of flow cytometry, fluorescence-activated cell sorting (FACS), magnetic-activated cell sorting (MACS), and/or affinity chromatography. In some embodiments, one or more methods of characterizing engineered activated B lineage cell subpopulations are employed during each step of a controlled cooling method.

In some embodiments, methods disclosed herein are used to segregate and/or characterize cryopreserved plasmablast cell subpopulations within a cryopreserved B lineage cell population. In some embodiments, plasmablast cell subpopulations are characterized through one or more of flow cytometry, fluorescence-activated cell sorting (FACS), magnetic-activated cell sorting (MACS), and/or affinity chromatography. In some embodiments, one or more methods of characterizing plasmablast cell subpopulations are employed during each step of a controlled cooling method. In some embodiments, methods disclosed herein are used to segregate and/or characterize cryopreserved engineered plasmablast cell subpopulations. In some embodiments, engineered plasmablast cell subpopulations are characterized through one or more of flow cytometry, fluorescence-activated cell sorting (FACS), magnetic-activated cell sorting (MACS), and/or affinity chromatography. In some embodiments, one or more methods of characterizing engineered plasmablast cell subpopulations are employed during each step of a controlled cooling method.

In some embodiments, methods disclosed herein are used to segregate and/or characterize cryopreserved plasma cell precursor subpopulations within a cryopreserved B lineage cell population. In some embodiments, plasma cell precursor subpopulations are characterized through one or more of flow cytometry, fluorescence-activated cell sorting (FACS), magnetic-activated cell sorting (MACS), and/or affinity chromatography. In some embodiments, one or more methods of characterizing plasma cell precursor subpopulations are employed during each step of a controlled cooling method. In some embodiments, methods disclosed herein are used to segregate and/or characterize cryopreserved engineered plasma cell precursor subpopulations. In some embodiments, engineered plasma cell precursor subpopulations are characterized through one or more of flow cytometry, fluorescence-activated cell sorting (FACs), magnetic-activated cell sorting (MACs), and/or affinity chromatography. In some embodiments, one or more methods of characterizing engineered plasma cell precursor subpopulations are employed during each step of a controlled cooling method.

In some embodiments, methods disclosed herein are used to segregate and/or characterize cryopreserved plasma cell subpopulations within a cryopreserved B lineage cell population. In some embodiments, plasma cell subpopulations may be characterized through one or more of flow cytometry, fluorescence-activated cell sorting (FACS), magnetic-activated cell sorting (MACS), and/or affinity chromatography. In some embodiments, one or more methods of characterizing plasma cell subpopulations are employed during each step of a controlled cooling method. In some embodiments, methods disclosed herein are used to segregate and/or characterize cryopreserved engineered plasma cell subpopulations. In some embodiments, engineered plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered plasma cell subpopulations are employed during each step of a controlled cooling method.

In some embodiments, methods disclosed herein are used to segregate and/or characterize cryopreserved short-lived plasma cell populations within a cryopreserved B lineage cell population. In some embodiments, short-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing short-lived plasma cell subpopulations are employed during each step of a controlled cooling method. In some embodiments, methods disclosed herein are used to segregate and/or characterize cryopreserved engineered short-lived plasma cell subpopulations. In some embodiments, engineered short-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered short-lived plasma cell subpopulations are employed during each step of a controlled cooling method.

In some embodiments, methods disclosed herein may be used to segregate and/or characterize cryopreserved long-lived plasma cell subpopulations with a B lineage cell population. In some embodiments, long-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing long-lived plasma cell subpopulations are employed during each step of a controlled cooling method. In some embodiments, methods disclosed herein are used to segregate and/or characterize cryopreserved engineered long-lived plasma cell subpopulations. In some embodiments, engineered long-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered long-lived plasma cell subpopulations are employed during each step of a controlled cooling method.

### EXEMPLIFICATION

### Example 1: Materials and Methods

The present Example demonstrates the cryopreservation methods that may be implemented to cryopreserve certain cell populations, including, but not limited to, B lineage cell populations. Initially, certain cell populations (e.g., B lineage cell populations) are cultured and expanded to about 500×10⁶ cells. Cell populations can comprise or be B lineage cell populations. B lineage cell populations comprise or be naïve B cells. B lineage cell populations comprise or be activated B lineage cells. B lineage cell populations may comprise or be plasmablast. B lineage cell populations may comprise or be plasma cells.

B lineage cell populations are transferred into a culture bag (e.g., VueLife bag). B lineage cell populations then undergo culture washes using Sepax CultureWash.

Next, B lineage cell populations undergo Ficoll clean-up and isolation. Such Ficoll clean-up and isolation may occur through the use of the NeatCell procedure.

B lineage cell populations are transferred from a culture bag (e.g., VueLife bag) into a centrifuge tube (e.g., 50 mL centrifuge tube). Next, these B lineage cell populations are transferred into vials and subsequently undergo centrifugation. After centrifugation, post-Ficoll product supernatant is removed. Next, the B lineage cell populations are resuspended in 5% HSA in PlasmasLyte-A. Then, the viable cell concentration is obtained and calculated by use of a cell counter for the B lineage cell population suspension. Once the cell count is taken, the B lineage cell population are then further formulated by addition of HSA in plasmalyte to a final concentration about 30×10⁶ cells/mL. Then, CryoStor 10 (CS10) is added to the B lineage cell population to result in a final cryopreservation preparation comprising 2.5% HSA in 50% PlasmaLyte-A with 50% CS10, and target concentration of 15×10⁶ cells/mL.

In some embodiments, B lineage cell populations are resuspended in cryopreservation media to a target concentration about 15×10⁶ cells/mL.

A sample of B lineage cells are then prepared by the addition of 2 mL of cryopreservation media (e.g., 2.5% HSA in PlasmaLyte-A with 50% CS10). Next, the sample of B lineage cells are placed in a controlled rate freezer and undergo the following controlled cooling steps in order to generate a cryopreserved B lineage cell preparation:
(i) cooling the chamber until the cryopreservation cell preparation reaches 4° C.;
(ii) cooling the chamber at a rate of 1° C./minute until the cryopreservation cell preparation reaches −4° C.;
(iii) cooling the chamber at a rate of 25° C./minute until the chamber reaches −40° C.;
(iv) heating the chamber at a rate of 10° C./minute until the chamber reaches −12° C.;
(v) cooling the chamber at a rate of 1° C./minute until the chamber reaches −40° C.;
(vi) cooling the chamber at a rate of 10° C./minute until the chamber reaches −90° C.

B lineage cell preparations are then subsequently transferred into a liquid nitrogen storage transfer box (e.g., cryopod) before being stored in an ultra-cold freezer (e.g., liquid nitrogen storage).

The methods described above may be performed on an engineered B lineage cell population resulting in a cryopreserved engineered B lineage cell preparation. Cryopreservation media can include about 0.5-5% HSA, 2-5% DMSO, 25-100% PlasmaLyte A, 25-100% CS10, and/or any combination thereof. Cryopreserved engineered B lineage cell preparations can comprise cell populations that have been previously frozen at −170° C. in media comprising one or more cryopreservation agents including HSA, PlasmaLyte, and CryoStor 10 (CS10). The concentration of HSA was 2.5%. The concentration of Plasmalyte was 50%. The concentration of CS10 was 50%. Cryopreserved engineered B lineage cell preparations can comprise engineered B lineage cell populations that have been previously frozen at −170° C. through one or more steps of controlled temperature changes in media comprising 2.5% HSA, 50% plasmalyte, and 50% CS10.

### Elisa to Detect Human Fix From Engineered B Lineage Cell Population

Engineered B lineage cell population supernatant was harvested from relevant experiments and frozen at −80° C. prior to testing. Samples were then tested for concentrations of human Factor IX (hFIX) in the 96 well FIX ELISA (Abcam-ab108831). Briefly, all kit components, samples, and standards were thawed and allowed to equilibrate to room temperature. 50 μL of sample or standard were added per well of pre-coated plates and then incubated at room temperature for 2 hours. After incubation, plates were washed 5 times with 200 μL of 1× Wash Buffer. Next, 50 μL of 1× biotinylated FIX detector antibody was added into each well and incubated for 1 hour at room temperature. Plates were then washed and 50 μL of 1× SP conjugate was added to each well. After addition of 1× SP conjugate, plates were then incubated for 30 minutes at room temperature. After another round of plate washing, 50 μL of Chromogen Substrate was added per well and incubated for 10 minutes at room temperature. After color develops in the wells, 50 μL of Stop Solution was added to quench. The plate absorbance was read at 450 nm and 570 nm by a Cytation5 Plate Reader. Sample values were interpolated using the standards at known hFIX concentrations.

### Flow Cytometry Analysis

Following resuspension of the cell samples, 2×10⁵ cells were set-aside for flow cytometric analysis. Samples were washed and stained with antibody cocktails focused on immune cell purity (markers: CD19, CD20, CD16, CD56, CD3, CD14, CD45), on B lineage cell phenotyping (markers: CD19, CD20, CXCR4, CD27, CD38, CD138), or on Ig profiling (markers: CD38, CD138, IgM, IgG, IgE, IgA). Cells were washed once in PBS, then stained with LIVE/DEAD Fixable Near-IR Dead Cell Stain (Life Technologies) for 10 minutes at room temperature. Cells were washed with Flow Cytometry Staining Buffer (eBioscience) and resuspended in Human TruStain FcX (BioLegend). After 5 minutes of Fc blocking at 4° C., samples were then resuspended in 100 μL of antibody cocktail master mix. Samples were incubated for 20 minutes at 4° C. in the dark, then washed two times before being resuspended in 400 μL staining buffer for acquisition on the flow cytometer. Single color controls for voltage gating and compensation were also prepared. Following compensation as guided by the NovoCyte software prompt, >10,000 cellular events were acquired per sample. Files were saved and imported into FlowJo. Live cell singlets were further gated in this software, then population percentages, total events, and geometric mean fluorescence intensity values were exported, formatted, and statistically analyzed.

For IgM/IgG profiling, intracellular staining was performed using cell fixation and permeabilization. Following the last wash from viability staining, cells were suspended in fixation buffer (4% paraformaldehyde) and incubated at room temperature for 20 minutes. Cells were then washed in Permeabilization Buffer (eBioscience) three times, then resuspended in Ig profiling antibody master mix (dilutions used were manufacturer's recommendation). Samples were incubated 20 minutes at room temperature protected from light, then washed 2 times with permeabilization buffer. Pellets were suspended in staining buffer as for fresh cell staining and run with forward/side-scatter gating adjusted for cell shrinkage after fixation.

### Example 2: Comparison of Fresh and Previously Frozen Cell Preparations In Vivo

The present Example demonstrates that fresh engineered cell preparations exhibit greater transgene expression as compared to previously frozen engineered cell preparations that were frozen in identical media conditions.

Naïve B cells from two different donors (LKP13 and LKP42) were isolated, activated, engineered, expanded, and differentiated as described above in Example 1. B lineage cells were engineered to express luciferase from the endogenous CCR5 locus. After Ficoll clean-up and isolation, cell populations were transferred into a base cell culture media and split into two groups. The first group (fresh engineered cell preparation) was administered to mice immediately. The second group (frozen B lineage preparation) was transferred into media comprising 100% CS10 at a concentration of 15×10⁶ cells/mL, and were passively frozen for 24 hours at −80° C. in an ethanol based freezing container before being frozen at −170° C. in liquid nitrogen. These cells were then administered to mice after thawing.

Among other things, the present disclosure demonstrates that subjects treated with previously frozen cell preparations may demonstrate reduced transgene and/or antibody expression as compared to subjects treated with fresh engineered cell preparations. Previously frozen engineered cell preparations may demonstrate lower engraftment rates as compared to fresh engineered cell preparations comprising identical media conditions. The present disclosure seeks to address these challenges and concerns with the described methods for cryopreservation of engineered cell preparations (e.g., engineered B lineage cell preparations) described herein.

### Example 3: Improved Cell Viability After Using Cryopreservation Methods Described Herein

The present Example demonstrates that cryopreserved engineered cell preparations (e.g., engineered B lineage cell preparation) may demonstrate improved cell viability and/or density when cryopreserved through steps of controlled temperature changes in media comprising certain cryoprotectants.

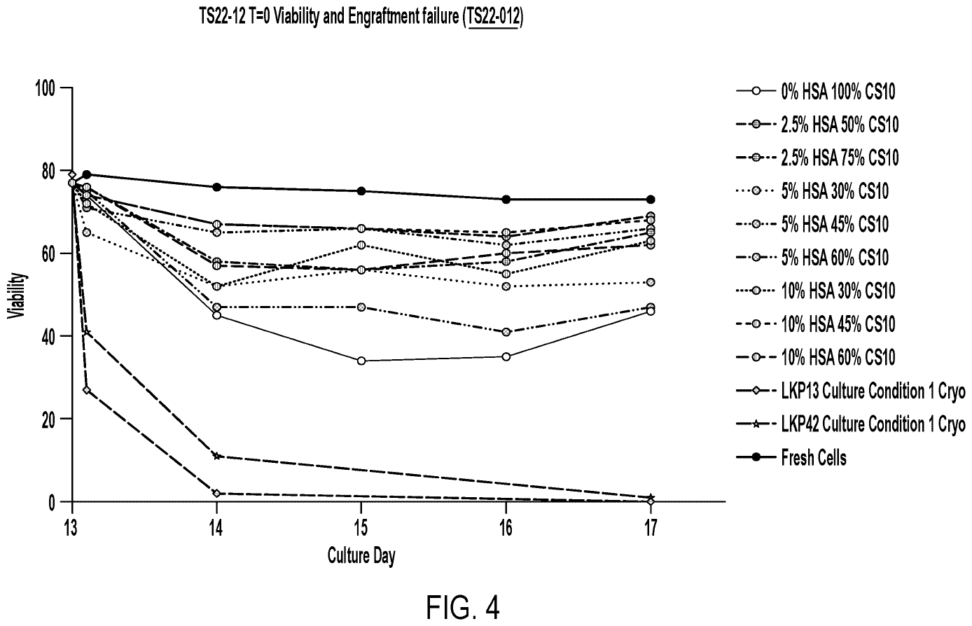

Naïve B cells from one donor (LKP22030) were isolated, activated, engineered, expanded, and differentiated as described above in Example 1. B lineage cells were engineered to express luciferase from the endogenous CCR5 locus. Cryopreserved B lineage cell preparations from two donors (LKP13 and LKP42), as described in Example 2 above, were used as a reference. After Ficoll clean-up and isolation, cell populations were split into groups, with each group placed in media comprising the cryoprotectants set forth in FIG. 4. Cell preparations were then cooled to −170° C. using the methods described in Example 1 above. Cryopreserved engineered B lineage cell preparations were then thawed and cultured in same media for an additional 1-5 days (days 14-18 of culture, in FIG. 4).

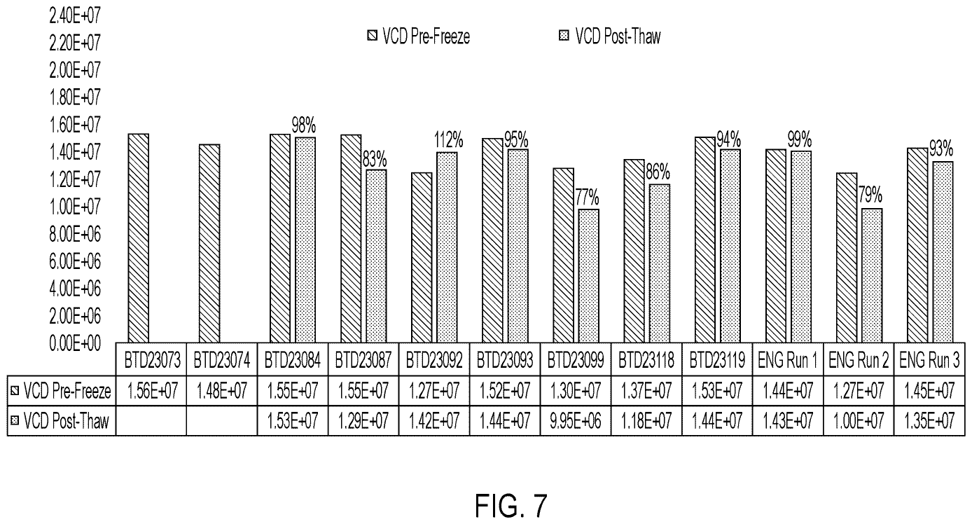

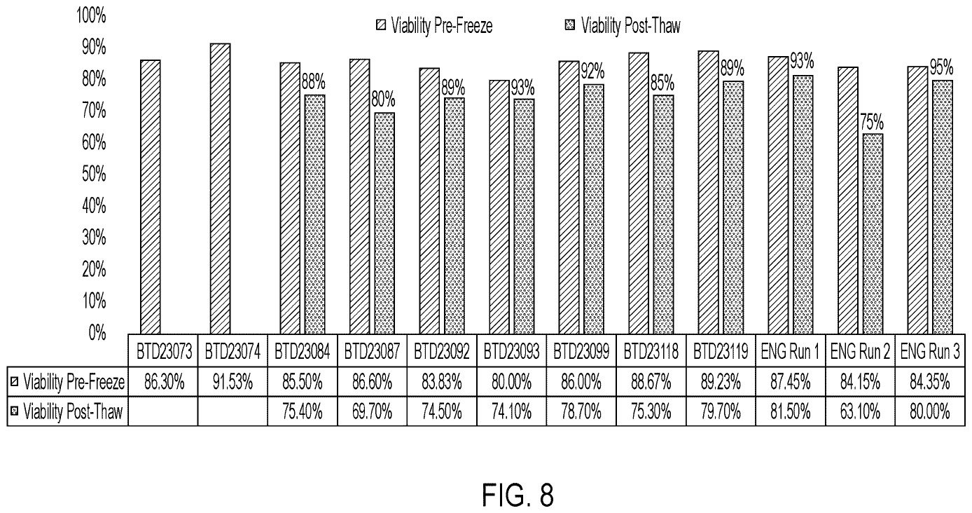

Additional B lineage cell preparations (BTD23073, BTD23074, BTD23084, BTD23087, BTD23092, BTD23093, BTD23099, BTD23118, BTD23119, ENG Run 1-3) were isolated, activated, engineered, expanded and differentiated as described herein. Cell number and viability via acridine orange (AO) and DAPI staining was measured in the various B lineage cell preparations prior to and after undergoing the cryopreservation methods described herein. Average cellular recovery and viability after thawing was approximately 92% and 88%, respectively (FIG. 7, FIG. 8).

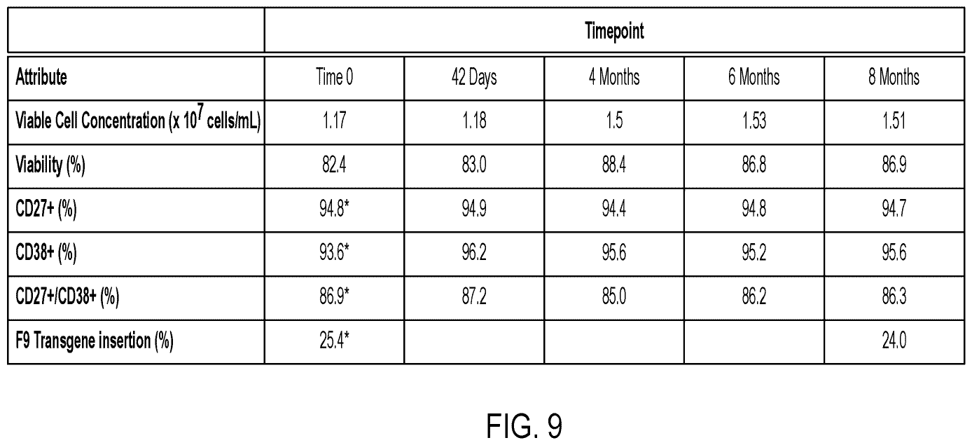

B lineage cell population was isolated, activated, engineered with human Factor IX expression cassette, expanded and differentiated as described above in Example 1. Engineered B lineage cell population then underwent the cryopreservation methods described above in Example 1. Viability and cell concentration was measured using AO/DAPI staining, whereas percentage of CD27, CD38, and CD27/CD38 expression was measured via flow cytometry at various time points before (Time 0) and after 42 days, 4 months, 6 months, and 8 months of cryopreservation (FIG. 9).

Among other things, the present disclosure demonstrates that engineered cell populations (e.g., engineered B lineage cell populations) cryopreserved through steps of controlled temperature changes in media comprising one or more cryoprotectants may provide improved cell viability and/or cell density after thawing as compared to a reference condition (e.g., media lacking one or more cryoprotectants). Engineered B lineage cell populations cryopreserved through steps of controlled temperature changes in media comprising one or more cryoprotectants may also provide improved cell viability for engineered B lineage cell populations as compared to a reference condition (e.g., media lacking one or more cryoprotectants). Engineered B lineage cell populations cryopreserved through steps of controlled temperature changes in media comprising between 2.5%-10% HSA and 4.5%-5% DMSO may provide improved cell viability after thawing and further culturing as compared to a reference condition (e.g., media lacking one or more cryoprotectants). Engineered B lineage cell populations cryopreserved through steps of controlled temperature changes in media comprising between 2.5%-10% HSA and 4.5%-5% DMSO may provide improved cell engraftment after administration to a subject, as compared to a reference condition (e.g., media lacking one or more cryoprotectants). populations cryopreserved through steps of controlled temperature changes in media comprising one or more cryoprotectants may also provide improved cell number for engineered B lineage cell populations as compared to a reference condition (e.g., media lacking one or more cryoprotectants).

### Example 4: Comparison of Fresh and Cryopreserved Engineered Cell Preparations In Vivo

The present Example demonstrates that cryopreserved engineered cell preparations (e.g., cryopreserved engineered B lineage cell populations) may demonstrate engraftment and/or transgene expression that is comparable and/or not significantly reduced as compared to fresh engineered cell preparations after administration to a subject. Subjects treated with cryopreserved engineered cell preparations described herein (e.g., cryopreserved engineered B lineage cell populations) may demonstrate persistent levels of transgene expression over an extended period of time (e.g., one or more weeks, one or more months, etc.)

Naïve B cells were isolated from different donors, activated, engineered, expanded, and differentiated as described above in Example 1. B lineage cells were engineered to express luciferase from the endogenous CCR5 locus. After Ficoll clean-up and isolation, B lineage cell populations were either placed in media comprising a base cell culture media and then directly administered to mice (fresh engineered cell preparations) or placed in media comprising 2.5% HSA, 50% PlasmaLyte, and 50% CS10, and frozen at −170° C. (cryopreserved engineered cell preparations). The first group (fresh engineered B lineage cell preparation) was administered to mice immediately. The second group (frozen B lineage cell preparation) was frozen at −170° C. and then administered to mice after thawing.

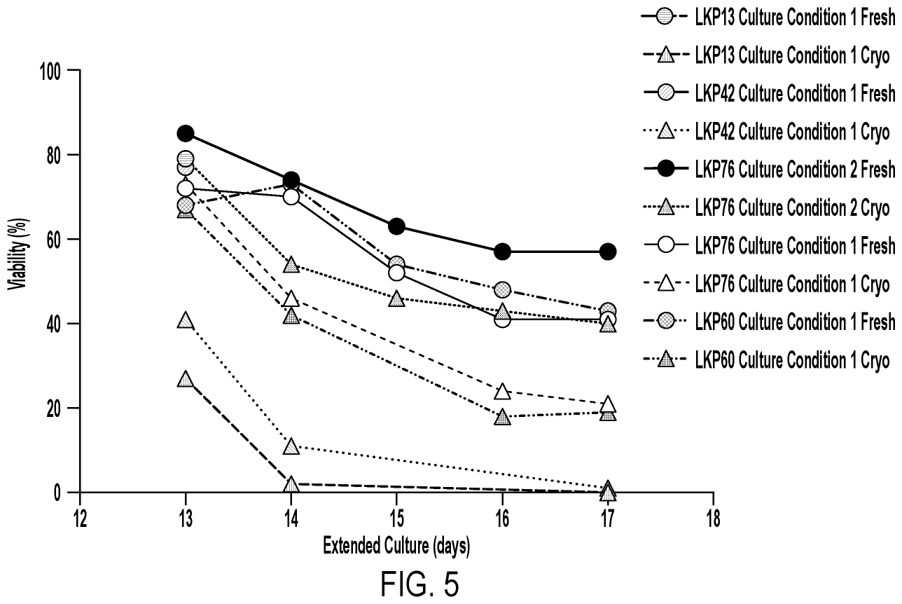

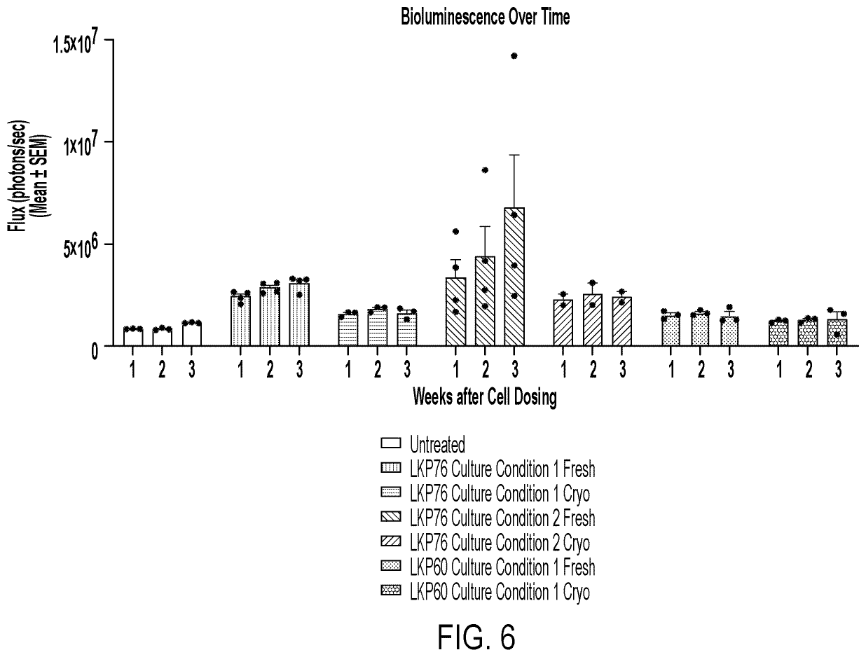

Whole-body imaging was conducted on mice at 1, 2, and 3 weeks post-administration of fresh and frozen engineered cell preparations to determine bioluminescence levels (FIG. 6). Cell viability among these donors, including those described in Example 2 above, were assessed and measured (FIG. 5).

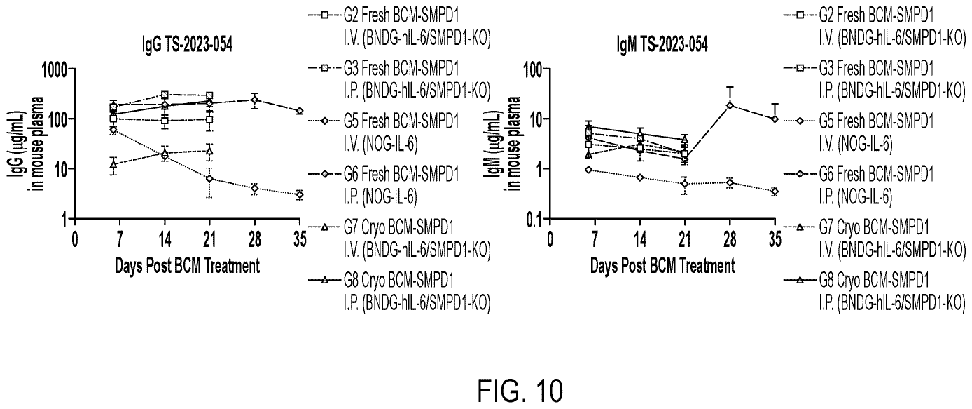

Additional Naïve B cell populations from different donors were isolated, activated, engineered, expanded, and differentiated as described above in Example 1. B lineage cells were engineered to express sphingomyelin phosphodiesterase 1 (SMPD1) from the endogenous CCR5 locus. After Ficoll clean-up and isolation, cell populations were transferred into a base cell culture media and split into 6 groups. The first four groups (fresh engineered cell preparations, “Fresh BCM-SMPD1”) was administered to mice immediately. The second set of three groups (frozen B lineage preparations, “Cryo BCM-SMPD1”) was transferred into media comprising 50% CS10 and 50% PlasmaLyte supplemented with HSA at a concentration of 15×10⁶ cells/mL, and were frozen via controlled rate freezer before being transferred to storage at −170° C. in liquid nitrogen. These cells were then administered to mice after thawing. Plasma samples were collected and levels of secreted IgG and IgM were quantified (FIG. 10).

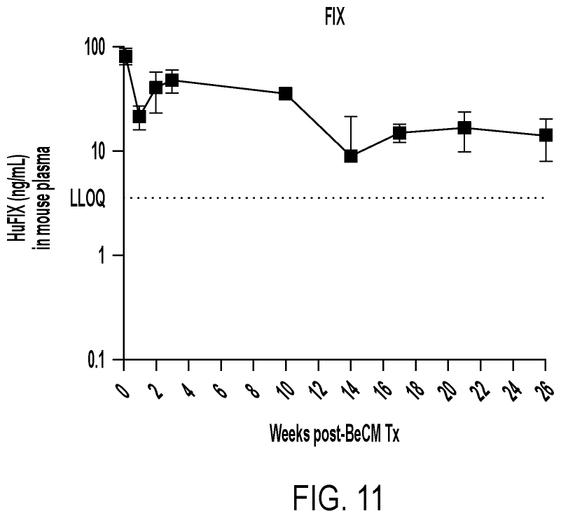

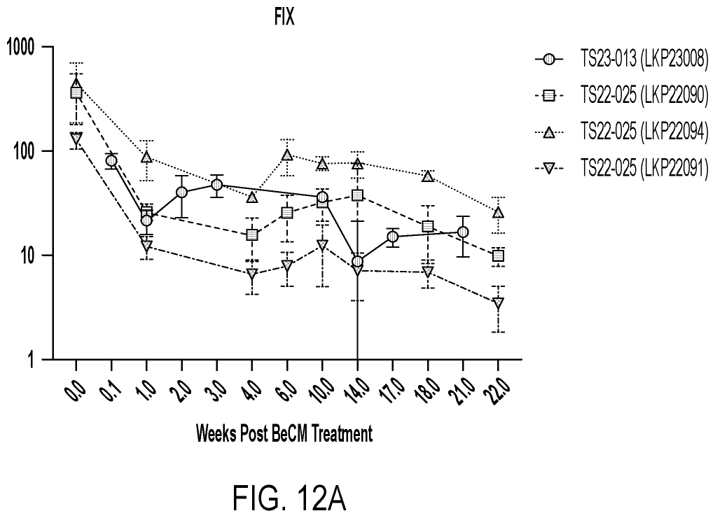

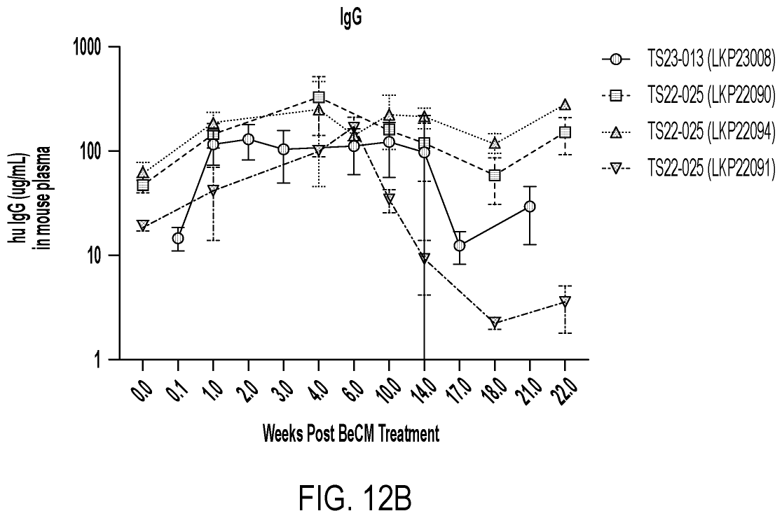

Further in vivo experiments were undertaken with B lineage cell populations that were isolated from different donors, activated, engineered, expanded, and differentiated as described above in Example 1. B lineage cells were engineered to express either human Factor IX (huFIX) from the endogenous CCR5 locus and preserved at −170° C. and then administered to mice after thawing using the methods described above in Example 1. Next, huFIX and human immunoglobulin G (huIgG) was measured from mouse plasma collected at various timepoints after administration (FIG. 11, FIG. 12A, and FIG. 12B).

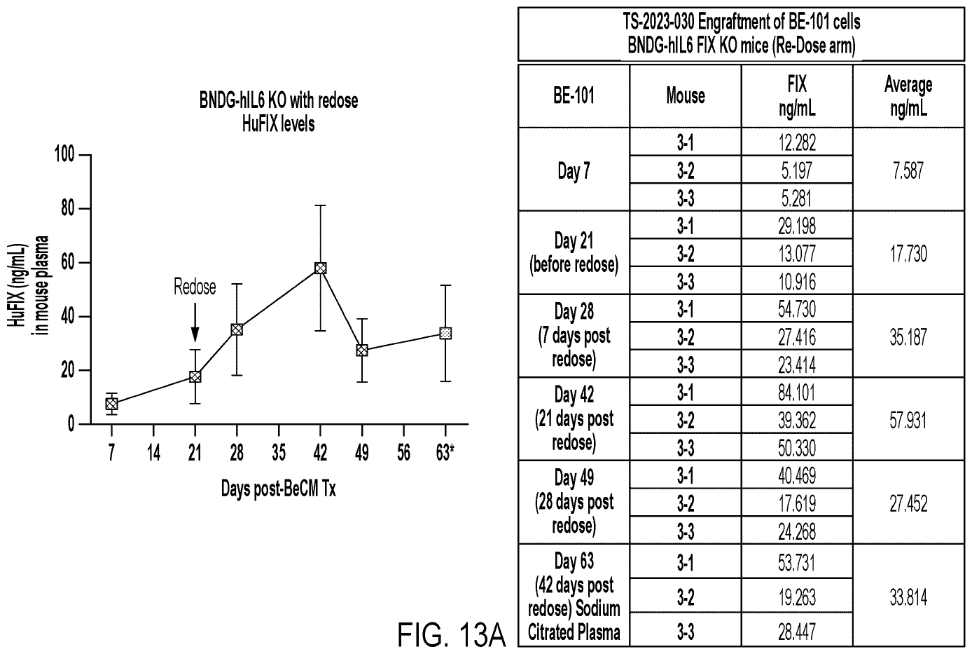

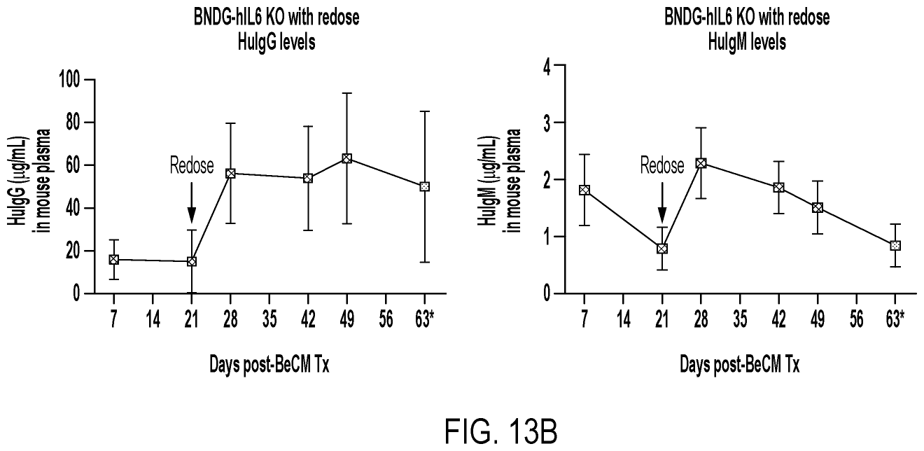

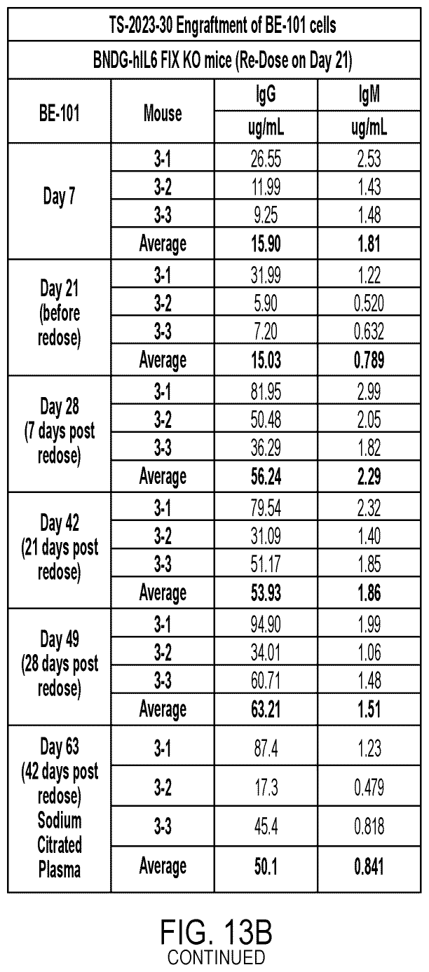

Additional in vivo experiment was undertaken on a mouse model using B lineage cells isolated, activated, engineered to express huFIX, expanded, differentiated, and cryopreserved as described above in Example 1. HuFIX Engineered B lineage cells were then administered to a mouse model with human interleukin 6 knockout (BNDG-hIL6 KO) and re-dosed with the engineered B lineage cell population on day 21. huFIX, huIgG, and human immunoglobulin M was measured after initial administration and after re-dosing (FIG. 13A and FIG. 13B).

Among other things, the present disclosure demonstrates that cryopreserved engineered cell preparations described herein (e.g., cryopreserved engineered B lineage cell populations) may provide engraftment and/or transgene expression that is comparable or not significantly reduced as compared to a reference condition (e.g., fresh engineered cell preparations) after administration to a subject. In some embodiments, cryopreserved engineered cell preparations described herein (e.g., cryopreserved engineered B lineage cell populations) may provide improved engraftment and/or transgene expression as compared to a reference condition (e.g., previously frozen engineered cell preparations lacking one or more cryoprotectants). In some embodiments, cryopreserved engineered cell preparations described herein (e.g., cryopreserved engineered B lineage cell populations) may provide transgene expression and stable cell populations for up to at least 22 weeks after administration as compared to a reference condition (e.g., previously frozen engineered cell preparations lacking one or more cryoprotectants). In some embodiments, cryopreserved engineered cell preparations described herein (e.g., cryopreserved engineered B lineage cell populations) may provide transgene expression and stable cell populations with one or more administrations of said cryopreserved engineered cell preparations (e.g., previously frozen engineered cell preparations lacking one or more cryoprotectants).

### REFERENCES

Balci and Can 2013
Cheng et al. 2022, “Ex Vivo Engineered Human Plasma Cells Exhibit Robust Protein Secretion and Long-Term Engraftment In Vivo,” Nature Communications.
Fluckinger et al. 1998
Hammerland et al. 2017
Jeske et al. 2021, “Vector Strategies to Actualize B Cell-Based Gene Therapies,” Journal of Immunology.
Jourdan et al. 2009, “An In Vitro Model of Differentiation of Memory B Cells into Plasmablasts and Plasma Cells Including Detailed Phenotypic and Molecular Characterization,” Immunobiology.
Khodadadi et al. 2019, “The Maintenance of Memory Plasma Cells,” Frontiers in Immunology.
Mazur et al. 2004
Meneghel et al. 2020, “Cryopreservation as a Key Element in the Successful Delivery of Cell-Based Therapies-A Review,” Frontiers in Medicine.
Murray et al. 2022, “Chemical Approaches to Cryopreservation,” Nature Reviews.
Nguyen et al 2019, “Factors Affecting Early Antibody Secreting Cell Maturation Into Long-Lived Plasma Cells,” Frontiers in Immunology.
Nutt et al. 2015, “The generation of antibody-secreting plasma cells,” Nature Reviews Immunology.
Rawlings et al. 1995
Rawlings et al. 1997
Radbruch et al., 2006, “Competence and competition: the challenge of becoming a long-lived plasma cell,” Nature Reviews Immunology.
Sanza et al. 2019, “Challenges and Opportunities for Consistent Classification of Human B Cell and Plasma Cell Populations,” Frontiers in Immunology.
Slifka et al. 2018, “Humoral Immunity Due to Long-Lived Plasma Cells,” Immunity.
Ticha et al. 2021, “Effects of Long-Term Cryopreservation of PBMC on Recovery of B Cell Subpopulations,” Journal of Immunological Methods
Whaley et al. 2021
WO2018/170150, “Engraftable Cell-Based Immunotherapy for Long-Term Delivery of Therapeutic Proteins.”

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice are hereby incorporated by reference in their entireties.

We claim:

1. A method of preparing a B lineage cell population for cryopreservation, the method comprising a step of:
contacting the B lineage cell population with cryopreservation media comprising:
about 4.5% to about 5% DMSO; and
about 2.5% to about 10% HSA;
thereby producing a cryopreservation cell preparation.

2. The method of claim 1, wherein the cryopreservation cell preparation undergoes controlled cooling in a controlled cooling chamber, comprising one or more steps of:
(i) cooling the chamber until the cryopreservation cell preparation reaches 4° C.;
(ii) cooling the chamber at a rate of 1° C./minute until the cryopreservation cell preparation reaches −4° C.;
(iii) cooling the chamber at a rate of 25° C./minute until the chamber reaches −40° C.;
(iv) heating the chamber at a rate of 10° C./minute until the chamber reaches −12° C.;
(v) cooling the chamber at a rate of 1° C./minute until the chamber reaches −40° C.;
(vi) cooling the chamber at a rate of 10° C./minute until the chamber reaches −90° C.

3. The method of claim 1 or 2, wherein the B lineage cell population is or comprises a population of genetically modified plasma cells.

4. The method of claim 1 or 2, wherein the B lineage cell population is or comprises a population of plasmablasts.

5. The method of claim 4, wherein the B lineage cell population is or comprises a population of plasma cell precursors.

6. The method of any one of claims 1-5, wherein the cryopreservation media comprises 5% DMSO.

7. The method of any one of claims 1-6, wherein the cryopreservation media comprises 2.5% HSA.

8. The method of any one of claims 1-7, wherein the cryopreservation cell preparation maintains at least 50% viability after being frozen for at least 24 hours.

9. The method of claim 8, wherein the cryopreservation cell preparation maintains at least 50% viability after being frozen for one week.

10. The method of claim 8, wherein the cryopreservation cell preparation maintains at least 50% viability after being frozen for one month.

11. The method of claim 8, wherein the cryopreservation cell preparation maintains at least 50% viability after being frozen for six months.

12. A composition comprising:
(i) a population of frozen, genetically modified B lineage cells; and
(ii) cryopreservation media comprising:
about 4.5% to about 5% DMSO; and
about 2.5% to about 10% HSA.

13. The composition of claim 12, wherein the cryopreservation media comprises 5% DMSO.

14. The composition of claim 12 or 13, wherein the cryopreservation media comprises 2.5% HSA.

15. The composition of any one of claims 12-14, wherein the transgene is Factor IX.

16. The composition of any one of claims 12-15, wherein the endogenous gene locus is CCR5 or JCHAIN.

17. The composition of any one of claims 12-16, wherein the genetically modified B lineage cells have been frozen for at least 24 hours and maintain at least 50% viability after thawing.

18. The composition of any one of claims 12-17, wherein the genetically modified B lineage cells have been frozen for at least 24 hours and at least 25% of cells secrete IgG after thawing.

19. The composition of any one of claims 12-18, wherein the genetically modified B lineage cells have been frozen for at least 24 hours and at least 25% of cells express IgM after thawing.

20. The composition of any one of claims 12-19, wherein the population has been frozen for at least one week.

21. The composition of any one of claims 12-20, wherein the population has been frozen for at least one month.

22. The composition of any one of claims 12-21, wherein the population has been frozen for at least six months.

23. A method of treating a patient for a disease, wherein the method comprises administering a population of genetically modified B lineage cells that have been cryopreserved.

24. The method of claim 23, wherein the population of genetically modified B lineage cells have been cryopreserved in media comprising about 4.5% to about 5% DMSO and about 2.5% to about 10% HSA and thawed prior to administration to a patient.

25. The method of claim 24, wherein the media comprises 5% DMSO.

26. The method of any one of claims 23-25, wherein the media comprises 2.5% HSA.
